(19) United States
(12) Patent Application Publication
(10) Pub. No.: US 2003/0044847 A1
(43) Pub. Date: Mar. 6, 2003

(54) Methods for anlyzing interactions between proteins in live and intact cells

(76) Inventors: Sidney Pestka, North Caldwell, NJ (US); Christopher D. Krause, Brick, NJ (US)

Correspondence Address: ROPES & GRAY, ONE INTERNATIONAL PLACE, BOSTON, MA, 02110-2624, US

(21) Appl. No.: 10/147,335

(22) Filed: May 15, 2002

Related U.S. Application Data
(60) Provisional application No. 60/291,119, filed on May 15, 2001.

(51) Int. Cl. G01N 33/53; G01N 33/567

(52) U.S. Cl. 435/7.1; 435/7.21

(57) ABSTRACT

The present invention describes a method for detecting the interaction of at least one intracellular protein and an extracellular protein using fluorescent markers and an FRET system. The method can be used to elucidate biological pathways and to evaluate potential drug candidates of therapeutic interest.

### RELATION TO OTHER APPLICATIONS

This Application claims priority to U.S. Provisional Application No. 60/291,119, filed on May 15, 2001, the entire content of which is incorporated herein by reference.

### BACKGROUND OF THE INVENTION

Specific protein-protein interactions are fundamental to most cellular functions. Polypeptide interactions are involved in, inter alia, formation of functional transcription complexes, signal transduction pathways, cytoskeletal organization (e.g., microtubule polymerization), polypeptide hormone receptor-ligand binding, organization of multi-subunit enzyme complexes, and the like.

Investigation of protein-protein interactions under physiological conditions has been problematic. Considerable effort has been made to identify proteins that bind to proteins of interest. Typically, these interactions have been detected by using co-precipitation experiments in which an antibody to a known protein is mixed with a cell extract and used to precipitate the known protein and any proteins which are stably associated with it. This method has several disadvantages, such as: (1) it only detects proteins which are associated in cell extract conditions rather than under physiological, intracellular conditions, (2) it only detects proteins which bind to the known protein with sufficient strength and stability for efficient co-immunoprecipitation, (3) may not be able to detect oligomers of the target, (4) it fails to detect associated proteins which are displaced from the known protein upon antibody binding, and (5) it does not yield data in cells in real time. Additionally, the precipitation techniques at best provide a molecular weight as the sole identifying characteristic. For these reasons and others, improved methods for identifying proteins which interact with a known protein have been developed.

One approach has been to use a so-called interaction trap system (also referred to as the “two-hybrid assay”) based in yeast to identify polypeptide sequences which bind to a predetermined polypeptide sequence present in a fusion protein (Fields and Song (1989) Nature 340:245). This approach identifies protein-protein interactions in vivo through reconstitution of a eukaryotic transcriptional activator.

The interaction trap systems of the prior art are based on the finding that most eukaryotic transcription activators are modular. Brent and Ptashne showed that the activation domain of yeast GAL4, a yeast transcription factor, could be fused to the DNA binding domain of E. coli LexA to create a functional transcription activator in yeast (Brent et al. (1985) Cell 43:729-736). There is evidence that transcription can be activated through the use of two functional domains of a transcription factor: a domain that recognizes and binds to a specific site on the DNA and a domain that is necessary for activation. The transcriptional activation domain is thought to function by contacting other proteins involved in transcription. The DNA-binding domain appears to function to position the transcriptional activation domain on the target gene that is to be transcribed. These and similar experiments (Keegan et al. (1986) Science 231:699-704) formally define activation domains as portions of proteins that activate transcription when brought to DNA by DNA binding domains. Moreover, it was discovered that the DNA binding domain does not have to be physically on the same polypeptide as the activation domain, so long as the two separate polypeptides interact with one another (Ma et al. (1988) Cell 55:443-446).

Fields and his coworkers made the seminal suggestion that protein interactions could be detected if two potentially interacting proteins were expressed as chimeras. They devised a method based on the properties of the yeast Gal4 protein, which consists of separable domains responsible for DNA-binding and transcriptional activation. Polynucleotides encoding two hybrid proteins, one consisting of the yeast Gal4 DNA-binding domain fused to a polypeptide sequence of a known protein and the other consisting of the Gal4 activation domain fused to a polypeptide sequence of a second protein, are constructed and introduced into a yeast host cell. Intermolecular binding between the two fusion proteins reconstitutes the Gal4 DNA-binding domain with the Gal4 activation domain, which leads to the transcriptional activation of a reporter gene (e.g., lacZ, HIS3) which is operably linked to a Gal4 binding site.

Yeast-based interaction trap systems in the art generally share common elements (Chien et al. (1991) PNAS 88:9578-82; Durfee et al. (1993) Genes & Development 7:555-69; Gyuris et al. (1993) Cell 75:791-803; and Vojtek et al. (1993) Cell 74:205-14). These systems use (1) a plasmid that directs the synthesis of a “bait”: a known protein which is brought to DNA by being fused to a DNA binding domain, (2) one or more reporter genes (“reporters”) with upstream binding sites for the bait, and (3) a plasmid that directs the synthesis of proteins fused to activation domains and other useful moieties (“prey”). Current systems direct the synthesis of proteins that carry the activation domain at the amino terminus of the fusion, facilitating the expression of open reading frames encoded by, for example, cDNAs.

Although most two hybrid systems use yeast, there are also mammalian variants. In one, interaction of activation tagged VPI6 derivatives with a Gal4-derived bait drives expression of reporters that direct the synthesis of Hygromycin B phosphotransferase, Chloramphenicol acetyltransferase, or CD4 cell surface antigen (Fearon et al. (1992) PNAS 89:7958-62). In the other, interaction of VP16-tagged derivatives with Gal4-derived baits drives the synthesis of SV40 T antigen, which in turn promotes the replication of the prey plasmid, which carries an SV40 origin (Vasavada et al. (1991) PNAS 88:10686-90).

Several industrially significant uses of two hybrid systems have emerged. One use is to identify new protein targets for pharmaceutical intervention. Typically, the two-hybrid method is used to identify novel polypeptide sequences which interact with a known protein (Silver et al. (1993) Mol. Biol. Rep. 17:155; Durfee et al. (1993) Genes Devel. 7:555; Yang et al. (1992) Science 257:680; Luban et al. (1993) Cell 73:1067; Hardy et al. (1992) Genes Devel. 6; 801; Bartel et al. (1993) Biotechniques 14:920; and Vojtek et al. (1993) Cell 74:205). Variations of the two-hybrid method have been used to identify mutations of a known protein that affect its binding to a second known protein (Li B and Fields S (1993) FASEB J 7:957; Lalo et al. (1993) PNAS 90:5524; Jackson et al. (1993) Mol. Cell. Biol. 13:2899; and Madura et al. (1993) J. Biol. Chem. 268:12046). Two-hybrid systems have also been used to identify interacting structural domains of two known proteins (Bardwell et al. (1993) Med. Microbiol. 8:1177; Chakraborty et al. (1992) J. Biol. Chem. 267:17498; Staudinger et al. (1993) J. Biol. Chem. 268:4608; and Milne et al. (1993) Genes Devel. 7:1755) or domains responsible for oligomerization of a single protein (Iwabuchi et al. (1993) Oncogene 8:1693; Bogerd et al. (1993) J. Virol. 67:5030). Variations of two-hybrid systems have been used to study the in vivo activity of a proteolytic enzyme (Dasmahapatra et al. (1992) PNAS 89:4159).

Although yeast two-hybrid system is a powerful method in delineating protein-protein interaction, significant drawbacks exist. For one thing, it is an artificial system in a heterologous environment, particularly, it depends on the fact that both proteins to be tested have to be in the nucleus for the transcription activation to occur. Other problems associated with the yeast two-hybrid system are described below. Thus, it is well known in the art that the technique is prone to false positive and false negative results. Therefore, what is needed are methods that can better mimic the physiological conditions normally present when authentic protein-protein interactions occur.

Measurement of protein-protein interaction in vivo by non-invasive techniques can help to validate the physiological significance of the interaction. This can also aid in identifying changes that occur in a cell or organism in response to physiological stimuli. One way to do this is through FRET (fluorescent resonance energy transfer). For example, cyclic AMP can be detected by FRET between separately labeled proteins that associate with each other but are not covalently attached to each other. See, U.S. Pat. No. 5,439,797. Likewise, calcium levels can be detected using a fluorescent indicator that includes a binding protein moiety, a donor fluorescent protein moiety, and an acceptor fluorescent protein moiety. The binding protein moiety has an analyte-binding region which binds an analyte and causes the indicator to change conformation upon exposure to the analyte. The donor fluorescent protein moiety is covalently coupled to the binding protein moiety. The acceptor fluorescent protein moiety is also covalently coupled to the binding protein moiety. In the fluorescent indicator, the donor moiety and the acceptor moiety change position relative to each other when the analyte binds to the analyte-binding region, altering fluorescence resonance energy transfer between the donor moiety and the acceptor moiety when the donor moiety is excited. See U.S. Pat. No. 6,197,928.

Gross structures (quaternary structures) of many multi-component membrane receptors may change upon activation. In fact, the quaternary structure and inter-relationship of many well-known receptor complexes are not known, either in their resting states (no ligand binding) or in their activated states (after ligand binding). In addition, observing FRET in live cells can creates some serious problems. For example, cells tend to cause light scattering, especially in populations of cells. If confocal microscope is not used, in many cases, the background is too high to allow meaningful or accurate measurements. Furthermore, spectrofluorimetry may be needed for spectral confirmation, so that specific fluorescence of certain proteins (such as GFP) can be differentiated from that of other cell components. And lastly, to the best of our knowledge, confocal microscope has never been coupled to monochrometer for these purposes.

It is accordingly an object of the invention to provide improved methods for detecting protein-protein interaction under physiological conditions in live and intact cells, preferably in single cells by using FRET (fluorescent resonance energy transfer).

### SUMMARY OF THE INVENTION

The present invention provides methods for detecting various protein-protein interactions under physiological conditions in live and intact cells. Particularly, the invention provides methods for detecting quaternary conformational change in multi-component membrane complexes, and methods to identify compounds or analytes that can bind to a multi-component membrane complex and induce its quaternary structural change, especially in high-throughput screening (HTS). These methods can also be extended to cell fragments or synthetic bilayers. In one embodiment, a multi-component membrane complex can be expressed in a host cell. For example, a first subunit of the complex is expressed in the host cell as a fusion protein with a donor fluorescent protein moiety, and a second subunit of the complex is expressed in the same cell as a fusion protein with a properly chosen acceptor fluorescent protein moiety. If the two subunits constitutively interact with each other in the live host cells under physiological conditions, FRET can be observed if the donor fluorescent protein moiety is activated by a laser beam of appropriate wave length emitted from a specially constructed confocal microscope. As a result, interaction between the two protein subunits can be detected by the presence of the FRET fluorescent signal. Upon binding of an analyte, such as a ligand of the multi-component membrane complex, quaternary structural changes can be detected by changes in the FRET signal, thereby detecting the binding of the analyte. Host cells exhibiting the FRET signal or losing the FRET signal before/after the analyte binding can be further isolated/recovered, if desired, by FACS sorting. The whole process can be streamlined and automated for high throughput screening (HTS).

The donor fluorescent protein moiety and the acceptor fluorescent protein moiety can be Aequorea-related fluorescent protein moieties. Preferably, in the case of mammalian cells, the donor fluorescent protein moiety is P4-3, EBFP, or WIB, and the acceptor fluorescent protein moiety is S65T, EGFP, or 10c (see Table 1). Other types of fluorescent protein moieties can be used for other types of host cells, such as bacterial cells.

The excitation wavelength of the donor fluorescent moiety can be one-photon, two-photon, or multiple photons for the purpose of reducing the background auto-fluorescence of host cells (see below).

The excitation light source or microscope should be compatible for the purpose of performing assays on live cells, i.e., it should be able to overcome the tremendous amounts of light scattering, and thus artifacts, generated by live cells. Preferably, it is an instrument coupling a confocal microscope with a spectrofluorimeter (see below). Fluorescence images are recorded on a confocal microscope based on substantial modifications done on a commercially available inverted microscope (Nikon, Diaphot 300, objective Nikon FLUOR 40, numerical aperture=1.3, oil immersion) without the laser that is usually supplied with the Nikon microscope. A laser beam from a separately housed titanium-sapphire femtosecond pulsed mode-lock tunable infrared laser was directed into the confocal microscope to excite the BFP or other donor fluorescent moieties. To measure FRET between donor and acceptor fluorescent moieties in a cell, the cell is excited with 2-photon excitation (half the energy of a one-photon excitation) light directly, with the cell itself capturing two-photons to obtain excitation at the usual excitation wavelength of the donor. In some experiments to examine the spectrum of the donor directly, the infrared laser emitting two-photon light is used with a doubling crystal to generate one-photon light to excite the donor, but this is not used for measuring FRET between donor and acceptor. For excitation of acceptor in a cell, a continuous wave argon laser was used with a band pass filter which generates the light capable of exciting the acceptor directly. The cells are excited by linearly polarized light through the back-port of the microscope.

The cells are mounted on a closed loop scan unit (Queensgate, Ascot, U.K., S222, scanning area 35 μm×35 μm) controlled by a modified Nanoscope E controller (Digital Instruments, Santa Barbara, Calif.), and the emitted fluorescence is collected by a single photon-counting module (EG&G, Salem, Mass., SPCM-AQ-161) for fluorescence images or by a combination of a monochromator (Acton Research, Acton, Mass., model 150) and a backilluminated nitrogen cooled charge-coupled device camera (Princeton Instruments, Trenton, N.J.) for spectra. Fluorescence images are obtained in the same way as described previously (Bopp M A, Jia Y, Li L, Cogdell R J, Hochstrasser R M. “Fluorescence and photobleaching dynamics of single light-harvesting complexes,” Proc Natl Acad Sci U S A. 94:10630-10635, 1997; Bopp M A, Sytnik A, Howard T D, Cogdell R J, Hochstrasser R M. “The dynamics of structural deformations of immobilized single light-harvesting complexes,” Proc Natl Acad Sci U S A. 96:11271-11276, 1999).

For high-throughput screening, a standard flow cytometer can be modified with the lasers described above to analyze the FRET. For detecting the FRET between donor and acceptor fluorescent moieties, cells will be excited with two-photon excitation light from an infrared laser so that the two photon capture by the cells will yield an effective one-photon excitation and emission at the wavelength of the acceptor. In addition, other methods can be used for two-photon excitation in live cells that was used to obtain 3-D images (Bahlmann K, Jakobs S, Hell SW “4Pi-confocal microscopy of live cells,” Ultramicroscopy 87:155-164, 2001). The isolated cells then can be analyzed individually by PCR or other amplification techniques.

The method is also applicable to FACS units such as the Meridian instrument that have been developed to scan and sort cells on monolayers rather than in a flowing fluid. Host cells to be used can be any cell types. Preferably, they can be engineered to contain fluorescent moieties suitable for the assay. More preferably, it can be prokaryotic cells (bacteria), yeast, COS cell, HeLa cell, established cancer cell, cells isolated from patients with major human diseases such as cancer, diabetes, multiple sclerosis, etc.

In certain embodiments, interaction between the two test peptides, or quaternary structural changes within a multi-component membrane complex, may occur only after proper stimulation of the host cell. The physiological stimulation can be, but is not limited to, growth factors, cytokines, changes in pH, starvation, environmental stresses or combinations thereof. Stimulation of live cell by these stimuli can typically lead to changes in protein modification (such as phosphorylation) or activation status (such as the active GTP-bound form vs. the inactive GDP-bound form of small G proteins), thus allowing interactions or quaternary structural changes to occur in response to these stimulations. Such changes in protein-protein interactions in response to physiological stimuli are fundamental phenomena in signal transduction and form the basis for cells to sense and adapt to their environment. Alternatively, constitutive interaction between the two test peptides, or one state of a quaternary structure may be disrupted after exposing the host cell to such physiological stimuli.

The ability to conduct these experiments in live cells confers unique advantages over the traditional yeast two-hybrid assay and its derivative methods. For one thing, it allows observation of protein-protein interactions under physiological conditions in real time without the artificially imposed restraint of transcriptional activation in nucleus. Secondly, it allows observation of protein-protein interactions which can only occur after certain activation steps such as phosphorylation, a feature not possible in any yeast two-hybrid assay. Further, the FRET signal is very fast (seconds) when compared to yeast two-hybrid assay (days), thus it is more suitable for automated real-time high throughput screening. Lastly, due to the direct and instant nature of the FRET signal, it is possible to track protein-protein interaction in real time, capturing transient as well as stable interactions, rather than waiting for the reporter genes to act and just observing the end result. Any structure in the cell can be analyzed, such as the nucleus, cytoplasm, cell membranes, etc.

Another aspect of the present invention provides methods for automated high throughput screens (HTS) to identify pharmaceutical preparations for enhancing or disrupting certain protein-protein interactions or the signal transduction pathways associated with such interactions.

In one embodiment, the method can be used to identify or test pharmaceutical preparations that either promote or disrupt certain interactions between two known proteins, such as two subunits of a multi-component membrane complex, either with or without the presence of certain physiological stimuli. In that respect, a first test protein/subunit is covalently linked to a donor fluorescent protein moiety, and a second test protein/subunit is covalently linked to a properly chosen acceptor fluorescent protein moiety. If the two test proteins/subunits constitutively interact with each other in the live host cells under physiological conditions, FRET can be observed after activation of the donor fluorescent protein moiety by a laser beam of appropriate wave length. In a preferred embodiment, upon binding of a ligand or analyte, the quaternary structure of the multi-component membrane complex will change, resulting in changes in FRET signal. A large number of pharmaceutical preparations can be quickly tested on these FRET-signal bearing cells in an automated HTS so that anything specifically destroying the FRET signal can be readily identified. Alternatively, pharmaceutical preparations specifically promoting certain interactions between two known proteins can also be identified. These processes can be done with or without the presence of certain natural/physiological stimulations (such as growth factors), so that pharmaceutical preparations capable of overcoming or bypassing these stimulations can be identified.

In a preferred embodiment, such method can be used to identify pharmaceutical preparations that inactivate signal transduction pathways downstream of a constitutively active mutant receptor. Many cancers are caused or associated with constitutively active mutant growth factor receptors, many of which (such as the her2/neu EGFR family receptor tyrosine kinases) are multi-component membrane complexes. As a consequence of this uncontrolled activation, many downstream signaling pathways are activated, leading to uncontrolled cell proliferation and cancer progression. Therefore, it is desirable to treat these conditions by inhibiting either the receptor or its downstream signaling molecules. This can be accomplished by selecting two known components of the downstream signaling pathway which interact in the presence of the constitutively active mutant receptor. By tagging these two proteins with donor and acceptor fluorescent moieties, respectively, pharmaceutical preparations specifically modifying (increasing or decreasing) the FRET signal can be readily identified in an automated HTS.

Another aspect of the invention provides a business method, comprising methods for identifying and exploiting pharmaceutical preparations that specifically promote or disrupt certain protein-protein interactions, methods for producing and selling drug candidates identified using this approach, and the sale or licensing of the right to manufacture and sell said pharmaceutical preparations.

Yet another aspect of the invention provides a method for the determination of the interactions of proteins in a given proteome. In one respect, a library of proteins within a given genome can be introduced into a host cell population, in the form of fusion proteins with a certain donor (or acceptor) fluorescent protein, by way of overproduction in the host cells using certain mammalian expression techniques. A second library of all proteins can then be introduced into the said host cell population, in the form of fusion proteins with a certain acceptor (or donor) fluorescent protein. Interactions between proteins belonging to the said two groups, with or without stimuli, generate FRET signals which can be used to sort out cells containing such signals using a FACS machine. Genes within each collected single cell can then be identified by techniques well-known in the art such as single cell PCR.

Alternatively, a first library of all proteins within a given genome can be introduced into a host cell population, in the form of fusion proteins with a certain donor (or acceptor) fluorescent protein, by way of overproduction in the host cells using certain mammalian expression techniques. Any given protein of interest can then be produced in this library of cells as a fusion protein with an acceptor (or donor) fluorescent moiety. Interactions between the protein of interest and any protein belong to the said library, with or without stimuli, will generate FRET signals which can be used to sort out cells containing such signals using a FACS machine. Genes within each collected single cell can then be identified by techniques well-known in the art such as single cell PCR. By testing individual proteins within a given proteome, it is possible to construct a detailed protein-protein interaction map within that proteome.

Thus, one aspect of the invention provides a method to detect quaternary structural change in a multi-component membrane complex upon binding of an analyte, comprising: (a) providing a multi-component membrane complex which binds an analyte, comprising: (i) a first fusion protein comprising a first polypeptide and a donor fluorescent protein moiety, wherein the donor fluorescent protein moiety, when excited at a first excitation wavelength, fluoresces at a first emission wavelength; (ii) a second fusion protein comprising a second polypeptide and an acceptor fluorescent protein moiety, wherein the acceptor fluorescent protein moiety, when excited by said first emission wavelength, fluoresces at a second emission wavelength; wherein in the absence of said analyte, said donor and acceptor fluorescent protein moieties are in sufficient close proximity for FRET (fluorescent resonant energy transfer) to occur, and excitation with light at said first excitation wavelength produces emission at said second emission wavelength; (b) exciting said donor fluorescent protein moiety with said first excitation wavelength; (c) contacting said multi-component membrane complex with said analyte; (d) detecting emission of at least one of said first or second emission wavelength; wherein a shift from emission at said second emission wavelength to said first emission wavelength indicates that a quaternary structural change occurs in said multi-component complex upon binding of said analyte.

In one embodiment, the multi-component membrane complex is a receptor. For example, the receptor can be a cytokine receptor, such as IFN-gamma receptor.

In another embodiment, the multi-component membrane complex is: a growth factor receptor, a GPCR (G-Protein Coupled Receptor), an MIRR (Multisubunit Immune Recognition Receptor) receptor, or an orphan receptor.

In another embodiment, at least one of said first and second fusion proteins is a transmembrane protein.

In another embodiment, said donor and acceptor fluorescent protein moieties are Aequorea-related fluorescent protein moieties. For example, the Aequorea-related protein moieties can be selected from the proteins listed in Table I. In a preferred embodiment, the donor and acceptor fluorescent protein moieties are BFP and GFP, respectively.

In another embodiment, the donor fluorescent protein moiety is excited at a first excitation wavelength by two-photo excitation.

In another embodiment, the multi-component membrane complex is provided in a single live and intact cell. In a preferred embodiment, the multi-component membrane complex is provided as a plasma membrane complex in a single live and intact cell. In another preferred embodiment, the donor and acceptor fluorescent protein moieties are both intracellular or are both extracellular.

In another embodiment, the multi-component membrane complex is a multimeric complex containing more than one member for at least one type of subunits. In a preferred embodiment, the multi-component membrane complex contains more than one said first polypeptide, wherein one or more said first polypeptide is fused to said donor fluorescent protein moiety. In another preferred embodiment, the multi-component membrane complex contains more than one said second polypeptide, wherein one or more said second polypeptide is fused to said acceptor fluorescent protein moiety.

In another embodiment, the first and second polypeptides are the same.

In another embodiment, the first and second polypeptides are different.

In another embodiment, the first excitation wavelength is generated by a laser. In a preferred embodiment, the laser is generated from a confocal microscope.

In another embodiment, the first and second fusion proteins are provided by introducing into a cell nucleic acids that encode said first and second fusion proteins.

Another aspect of the invention provides a method of identifying a ligand for a multi-component membrane complex, comprising: (a) providing a multi-component membrane complex, comprising: (i) a first fusion protein comprising a first polypeptide and a donor fluorescent protein moiety, wherein the donor fluorescent protein moiety, when excited at a first excitation wavelength, fluoresces at a first emission wavelength; (ii) a second fusion protein comprising a second polypeptide and an acceptor fluorescent protein moiety, wherein the acceptor fluorescent protein moiety, when excited by said first emission wavelength, fluoresces at a second emission wavelength; wherein in the absence of said analyte, said donor and acceptor fluorescent protein moieties are sufficiently close for FRET (fluorescent resonant energy transfer) to occur, and excitation with light at said first excitation wavelength produces emission at said second emission wavelength; (b) exciting said donor fluorescent protein moiety with said first excitation wavelength; (c) contacting said multi-component membrane complex with a test compound; (d) detecting emission of at least one of said first or second emission wavelength; (e) identifying a test compound as the ligand of said multi-component membrane complex if a shift from emission at said second emission wavelength to said first emission wavelength occurs.

In one embodiment, the multi-component membrane complex is provided in a single live and intact cell. In a preferred embodiment, the multi-component membrane complex is provided as a plasma membrane complex in a single live and intact cell. In another preferred embodiment, the donor and acceptor fluorescent protein moieties are both intracellular or are both extracellular. In another embodiment, the method further comprises carrying out steps (a)-(e) in a microtiter plate comprising 96 wells, preferably 384 wells, or even 1536 wells. Other predetermined pattern of wells (other than grids or square-shaped) may also be used. In another embodiment, step (e) is effected by a FACS (Fluorescent Activated Cell Sorter) machine.

In another embodiment, the multi-component membrane complex is a receptor. For example, the receptor can be a cytokine receptor, such as IFN-gamma receptor. In a related embodiment, the receptor is: a growth factor receptor, a GPCR (G-Protein Coupled Receptor), an MIRR (Multisubunit Immune Recognition Receptor) receptor, or an orphan receptor.

In another embodiment, at least one of said first and second fusion proteins is a transmembrane protein.

In another embodiment, the donor and acceptor fluorescent protein moieties are Aequorea-related fluorescent protein moieties. In a preferred embodiment, the Aequorea-related protein moieties are selected from the proteins listed in Table I. In a most preferred embodiment, the donor and acceptor fluorescent protein moieties are BFP and GFP, respectively.

In another embodiment, the donor fluorescent protein moiety is excited at a first excitation wavelength by two-photo excitation.

In another embodiment, the multi-component membrane complex is a multimeric complex containing more than one member for at least one type of subunits. For example, the multi-component membrane complex may contain more than one said first polypeptide, wherein one or more said first polypeptide is fused to said donor fluorescent protein moiety. Alternatively, the multi-component membrane complex contains more than one said second polypeptide, wherein one or more said second polypeptide is fused to said acceptor fluorescent protein moiety.

In another embodiment, the first and second polypeptides are the same.

In another embodiment, the first and second polypeptides are different.

In another embodiment, the first excitation wavelength is generated by a laser. In a preferred embodiment, the laser is generated from a confocal microscope.

In another embodiment, the first and second fusion proteins are provided by introducing into a cell nucleic acids that encode said first and second fusion proteins.

Another aspect of the invention provides a method of conducting a pharmaceutical business, comprising: (a) identifying, using any of the suitable method recited above, a ligand for a multi-component membrane complex; (b) conducting therapeutic profiling of said ligand identified in step (a), or further analogs thereof, for efficacy and toxicity in animals; and (c) formulating a pharmaceutical preparation including one or more ligands identified in step (b) as having an acceptable therapeutic profile.

In another embodiment, the method further comprises a step of establishing a distribution system for distributing the pharmaceutical preparation for sale.

In another embodiment, the method further comprises a step of establishing a sales group for marketing the pharmaceutical preparation.

Another aspect of the invention provides a method of conducting a target discovery business comprising: (a) identifying, using any of the suitable method recited above, a ligand for a multi-component membrane complex; (b) (optionally) conducting therapeutic profiling of ligands identified in step (a) for efficacy and toxicity in animals; and (c) licensing, to a third party, the rights for further drug development and/or sales for agents identified in step (a), or analogs thereof.

Although the above described embodiments are mostly independent of one another, the invention also contemplates embodiments containing two or more of the features/embodiments described above.

Other features and advantages of the invention will be apparent from the following detailed description, and from the claims. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes 1-IV (D. M. Weir and C. C. Blackwell, eds., 1986); Manipulating the Mouse Embryo, (Cold Spring Harbor Laboratory Press, Cold Spring Harbor, N.Y., 1986). Methods in Enzymology, Vols. 78, 79 and 119, have information about the interferons and their use.

### BRIEF DESCRIPTION OF THE DRAWINGS

FIG. 1 Model of the IFN-γ receptor complex.

FIG. 2 Schematic of fluorescence transfer between blue and green fluorescent proteins fused to IFN-γR1 and IFN-γR2 chains. Blue and green fluorescent proteins, BFP and GFP, were fused to the IFN-γR1 and IFN-γR2 chains as described. Because the cellular background was high when the BFP was excited with blue light at 380 nm, infrared light at 760 nm was used to excite the BFP with two photons. With no fluorescence transfer, the excitation of the BFP will emit light with a maximum at 445 nm. If fluorescence transfer from BFP to GFP occurs, the maximum wavelength of the emitted light will be seen at 509 nm.

FIG. 3 Demonstration that fluorescent receptor chains are functional. The human receptor chains (IFN-γR1 and FL-IFN-γR2) without fluorescent proteins (left panel) and those with the fluorescent proteins (IFN-γR1/EBFP and FL-IFN-γR2/GFP) (right panel) were transfected into Chinese hamster ovary (CHO) q3 cells and MHC class I antigen induction measured in response to hamster and human IFN-γ. In the absence of the human receptor chains, Hu-IFN-γ has no effect on CHO cells (78, 87, 98). The hamster IFN-γ was used as a positive control to show that the CHO cells respond to hamster IFN-γ. The CHO q3 cells were derived as follows. Hamster UCH-12 cells containing a stable translocation of human chromosome 3q were obtained from Carol Jones and David Patterson. UCH-12 cells were transfected with plasmid pJYI50R1.1 that encodes the intact HLAB7 gene (101) and the resulting cells designated q3. This hamster cell line, derived from CHO-K1, does not express human (Hu−) IFN-γR1 or Hu-IFN-yR2, and can reconstitute signaling in response to Hu-IFN-γ only when both chains are expressed in these cells. The stably transfected population of q3 cells was grown in F12 media supplemented with 10% FBS without selecting antibiotics. Selection was performed by sorting of two percent of cells exhibiting the greatest fluorescence in an MHC assay after three days treatment with Hu-IFN-γ. After three rounds of selection, individual clones were isolated by limiting dilution and amplified. Clones were isolated that induced significantly higher levels of MHC Class I surface antigen in response to Hu-IFN-β. From these clones, one clone (designated q3) was chosen which possessed a high efficiency of transfection with a plasmid encoding IFN-γR1 and IFN-γR2 and exhibited expression of reconstituted IFN-γ receptor complexes assayed by MHC Class I surface antigen induction of the geneticin-resistant transfected population in response to Hu-IFN-γ. MHC class I antigen induction in response to IFN-γ was performed as described (93).

FIG. 4 The fluorescent receptor chains exhibit their characteristic fluorescent signatures: images and spectra of human IFN-γR2/GFP and IFN-γR2/EBFP transfected into cells. The FL-IFN-γR2/GFP and FL-IFN-γR2/EBFP were each separately transfected into COS cells. A camera was used with the confocal microscope to obtain the images for human IFN-γR2/GFP (top left) and IFN-γR2/EBFP (top right) in COS-1 cells. Similar images were obtained for IFN-γRI/BFP and IFN-γR1/EBFP (not shown). The spectral signatures of GFP (green) and BFP (blue) can be seen in COS—I cells expressing FL-IFN-γR2/GFP and FL-IL-10/γR2/BFP (lower left and right, respectively). The black curves represent the relative epifluorescence of the cells in the absence of the respective transfected fluorescent receptor chains.

FIG. 5 Illustration of matched (FL-IFN-γR2/GFP and IFN-γRI /EBFP) and mismatched (IFN-γRI/EBFP and FL-IL-10R2/GFP) pairs of receptors. The matched pair, FL-IFN-γR2/GFP and FL-IFN-γR1/EBFP, are the two chains of the IFN-γ receptor complex fused to GFP and EBFP, respectively. The mismatched pair, IFN-γR1/EBFP and FL-IL-10R2/GFP, are first chain of the IFN-γ receptor complex and second chain of the IL-10 receptor complex, fused to EBFP and GFP, respectively.

FIG. 6 Comparison of fluorescence emission spectra of cells expressing the matched and mismatched pair of receptor chains. The matched receptor chains are FL-IFN-γR2/GFP and IFN-γR1/EBFP (green curve); the mismatched receptor chains are IFN-γR1/EBFP and FL-IL-10R2/GFP (blue curve). The fluorescence emission spectra in response to two-photon excitation at 760 nm are shown.

FIG. 7 Fluorescence spectra of cells expressing the mismatched pair of receptor chains, IFN-γR1/EBFP and FL-IL-1 OR2/GFP, in presence (blue curve) and absence (red curve) of IFN-γ. The fluorescence emission spectra in response to two-photon excitation at 760 nm are shown.

FIG. 8 Comparison of fluorescence spectra of cells expressing the matched pair of receptor chains in presence and absence of IFN-γ. The matched receptor chains are FL-IFN-γR2/GFP and IFN-γRI/EBFP. The spectrum in green was taken in the absence of IFN-γ. IFN-γ (3500 units/ml) was then added to the medium and the spectrum taken (blue curve) of the same region in the same cell. The fluorescence emission spectra in response to two-photon excitation at 760 nm are shown.

FIG. 9 Fluorescence transfer between the FL-IFN-γR2/GFP and FL-IFN-γR2/EBFP chains. Both FL-IFN-γR2/GFP and FL-IFN-γR2/EBFP were transfected into COS-1 cells in the absence (left panels) or presence (right panels) of the unlabeled IFN-γR1 chain. Spectra were taken in the presence and absence of IFN-γ.

FIG. 10 Model of the change in receptor structure on engagement of the ligand IFN-γ. When IFN-γ binds to the receptor complex the distance between the IFN-γR1 and IFN-γR2 chains and the distance between the two IFN-γR2 chains increases.

FIG. 11 Schematic of the confocal microscope coupled to lasers, photon counter, monochromater and CCD camera. Either single photon excitation at 488 nm of the GFP with an argon laser delivering 0.5 μW at the sample or a pulsed femtosecond mode-locked infrared Ti:sapphire laser (2 mW) tuned to 760 nm was used. The lasers were directly coupled to the microscope before the lens LI. The blue line represents the path of the light for excitation of the sample placed on the scanning platform. The green line represents the light path of the emission from the sample on the scanning platform back through the objective lens, to the mirror (M5), then passing through the dichromatic mirror (DM) along the path (green line) to the photon counting detector (APD) to collect the images or to the monochromoter (Mono) and spectral detector (CCD) to analyze the spectra. L1-L4 represent lenses; NF, a neutral density filter; M1-M5, mirrors; F, a band pass filter; DM is a dichromatic mirror; OBJ, the objective lens; APD, a photon counting detector; A, the aperture; CCD, the spectral detector; and Mono, the monochromater. The scanning of the sample was done with a Princeton Instrument P-731 stage and controlled by a Digital instrument Nanoscope IIIA unit. M3 is a flipper mirror allowing the emission to reach either the CCD camera or the photon counting detector (APD).

FIG. 12 Shows a fluorescent emission spectra of a point in the cells taken before (red) and after (in blue) treatment with IFN-γ. The test cell expresses GFP-IFN-γR1 and BFP-IFN-γR2 receptors. The sample was excited at 760 nm to observe the FRET signal (red and blue), and at 488 nm to observe only the GFP signal (in green). FIG. 13 Depicts images of a single sample cell excited at 760 nm (two-photon excitation) taken prior to treatment with IFN-γ (top left), after addition of IFN-gamma (lower left), and excited at 488 nm after addition of IFN-γ (lower right) to show only the GFP signal.

### DETAILED DESCRIPTION OF THE INVENTION

I. Overview The eukaryotic interaction trap system (“ITS”), originally developed by Fields and Song (Nature (1989) 340:245) in yeast, is a powerful in vivo assay to detect protein-protein interactions. It has already had a large impact on basic and applied biological research. In industry, it is being used to isolate and characterize new targets for drug development. It permits researchers to isolate small organic molecules, peptides, and nucleic acids that may lead to new drugs. Future applications for genome characterization and for modulation of specific protein-protein interactions are on the horizon. The ramifications of this technology promise to be exciting.

The present invention makes available an improved method to detect protein-protein interaction in live cells under physiological conditions by taking advantage of the FRET technique. As described in the appended examples, one protein can be fused to a donor fluorescent moiety, while the other can be fused to an acceptor fluorescent moiety. If the two proteins interact in a live cell, a FRET signal is generated upon excitation of the donor fluorescent moiety with a confocal laser beam. The versatility of this system makes it generally suitable for many, if not all of the applications involving protein-protein interaction. Moreover, the ability to stimulate live cells under physiological conditions and observe FRET either before or after the stimulation, coupled with the speed of the system and capacity for automated HTS, confer the tremendous advantages of this invention over the traditional yeast two-hybrid systems.

Another benefit in the use of this method is that, in contrast to the eukaryotic two-hybrid system, nuclear localization of the bait and prey polypeptides is not a concern. Any region and structure of the cells can be examined.

Still another advantage of the use of the system can be realized where certain protein-protein interactions which depend on post-translational modifications, such as phosphorylation, can be readily detected in this system. The same interactions will almost certainly be missed by the traditional two-hybrid assay and will be much more laborious to perform with other techniques such as immunoprecipitation.

A method for detecting interactions between two polypeptides is provided in accordance with the present invention. The method generally includes, with some variations, providing a first protein expressed in the host cell as a fusion protein with a donor fluorescent protein moiety, and a second protein expressed in the same cell as a fusion protein with an acceptor (or donor) fluorescent protein moiety; activating the donor (or acceptor) fluorescent moiety in the live host cell under physiological conditions by a laser beam of appropriate wave length emitted from a specially constructed confocal microscope as described herein; and observing and recording the disruption or generation of the FRET signal. Host cells exhibiting the FRET signal can be further isolated/recovered, if desired, by FACS sorting. The whole process can be streamlined and automated for high throughput screening (HTS).

The cell can be engineered to include a first chimeric gene which is capable of being expressed in the host cell. The chimeric gene encodes a fusion protein which comprises (i) a donor fluorescent moiety, and (ii) a polypeptide for which complex formation is to be tested.

A second chimeric gene can also be provided in the cell, the second chimeric gene encoding a second fusion protein comprising (i) an acceptor fluorescent moiety, and (ii) a second polypeptide which is to be tested for interaction with the first polypeptide. In certain embodiments of the invention, the donor fluorescent protein moiety and the acceptor fluorescent protein moiety can be Aequorea-related fluorescent protein moieties. Preferably, for use in mammalian cells, the donor fluorescent protein moiety is P4-3, EBFP, or WIB, and the acceptor fluorescent protein moiety is S65T, EGFP, or 10c.

In one embodiment, both the first and the second chimeric genes are introduced into the host cell in the form of plasmids or vectors using well known transfection techniques.

The interaction, if any, between the first and second fusion proteins in the host cell can cause the donor fluorescent moiety to be recruited in close vicinity of the acceptor fluorescent protein moiety, which, upon activation of the donor fluorescent moiety by a laser emitted from a laser confocal microscope, generates the FRET signal. The method can be carried out by introducing the first and second chimeric genes into the host cell, and subjecting that cell to conditions under which the first and second hybrid proteins are expressed in sufficient quantity in the host cell. The formation of a complex between the two test proteins results in a detectable FRET signal. Accordingly, the formation of a complex between these two proteins, for example, can be detected, and FRET cells isolated, if desired, by FACS on the basis of evaluating the strength of the FRET signal. Meanwhile, the agents causing or disrupting such signals can also be identified, preferably in an automated HTS; and agents causing initiation of such signals can also be identified.

When the background cellular fluorescent signals are relatively low, the instant methods can be practiced in a homogeneous population of cells, with specifically chosen fluorescent proteins. However, in a preferred embodiment, the methods are practiced in single cells, using confocal microscope generated laser sources.

II. Definitions

Before further describing the invention, certain terms employed in the specification, examples and appended claims are, for convenience, collected here.

By “amplification” or “clonal amplification” is meant a process whereby the density of host cells having a given phenotype is increased.

By “analyte” is meant a molecule or ion in solution that binds to the binding protein. For example, it can be a candidate ligand for a multi-component membrane complex, such as IFN-gamma and IFN-gamma receptors.

A fluorescent protein is an “Aequorea-related fluorescent protein” if any contiguous sequence of 150 amino acids of the fluorescent protein has at least 85% sequence identity with an amino acid sequence, either contiguous or non-contiguous, from the wild type Aequorea green fluorescent protein. More preferably, a fluorescent protein is an Aequorea-related fluorescent protein if any contiguous sequence of 200 amino acids of the fluorescent protein has at least 95% sequence identity with an amino acid sequence, either contiguous or non-contiguous, from the wild type Aequorea green fluorescent protein. Similarly, the fluorescent protein can be related to Renilla or Phialidium wild-type fluorescent proteins using the same standards. Some Aequorea-related engineered versions described in Table I. Other variants or mutants are within the scope of the invention as described, for example, in the Examples.

By “binding protein” is meant a protein capable of binding an analyte. For example, a multi-component membrane complex (IFN-gamma receptor complex) is a binding protein for IFN-gamma (analyte).

“Close proximity” means that the two molecules concerned are separated only by a small distance, if not in direct contact with each other. For example, two proteins that bind to each other is in close proximity to each other. In a preferred embodiment, the small distance is no more than 20 nm, or no more than 10 nm. More preferably, it is no more than 5 nm, 2 nm, 1 nm or less.

By “covalently bonded” it is meant that two domains are joined by covalent bonds, directly or indirectly. That is, the “covalently bonded” proteins or protein moieties may be immediately contiguous or may be separated by stretches of one or more amino acids within the same fusion protein.

By a “DNA binding domain” or “DBD” is meant a polypeptide sequence which is capable of directing specific polypeptide binding to a particular DNA sequence (i.e., to a DBD recognition element). The term “domain” in this context is not intended to be limited to a discrete folding domain. Rather, consideration of a polypeptide as a DBD for use in the bait fusion protein can be made simply by the observation that the polypeptide has a specific DNA binding activity. DNA binding domains, like activation tags, can be derived from proteins ranging from naturally occurring proteins to completely artificial sequences.

By “fluorescent protein” is meant any protein capable of emitting light when excited with appropriate electromagnetic radiation/light. Fluorescent proteins include proteins having amino acid sequences that are either natural or engineered, such as the fluorescent proteins derived from Aequorea-related fluorescent proteins. Another type of fluorescent protein contains natural or synthetic amino acids, co-factors, or other adducts that are fluorescent.

In FRET, the “donor fluorescent protein moiety” and the “acceptor fluorescent protein moiety” are selected so that the donor and acceptor moieties exhibit fluorescence resonance energy transfer when the donor moiety is excited. One factor to be considered in choosing the donor/acceptor fluorescent protein moiety pair is the efficiency of FRET between the two moieties. Preferably, the efficiency of FRET between the donor and acceptor moieties is at least 10%, more preferably at least 50%, and most preferably at least 80%. The efficiency of FRET can be tested empirically using the methods known in the art. This also depends on distance between donor and acceptor, and thus distances can be measured between proteins. Mutant proteins can also be screened using this technique. Langois et al., Journal of Molecular Biology, Vol. 106, 297-313 (1976). R. Langois et al. Biochemistry, Vol. 16, 2349-2356 (1977).

As used herein, the terms “heterologous DNA” or “heterologous nucleic acid” is meant to include DNA that does not occur naturally as part of the genome in which it is present, or DNA which is found in a location or locations in the genome that differs from that in which it occurs in nature, or occurs extra-chromosomally, e.g., as part of a plasmid.

The terms “interactors”, “interacting proteins” and “candidate interactors” are used interchangeably herein and refer to a set of proteins which are able to form complexes with one another, preferably non-covalent complexes.

By “moiety” is meant a molecule (or a functional part of a molecule) that is attached to another molecule (or part thereof). Thus, a “fluorescent protein moiety” is a fluorescent protein (or a functional part) coupled to another protein or a linker sequence; and a “binding protein moiety” is a part of a binding protein coupled to a fluorescent protein moiety.

“Multi-component membrane complex” means a membrane complex containing more than one subunit or polypeptide. At least one of the subunits/polypeptides of the complex is a membrane-associated polypeptide, preferably through at least one transmembrane domain, although the membrane-associated polypeptide may contain multiple transmembrane domains. The other component subunits of the complex at least are capable of associating with the membrane-associated subunit, for example, after binding of the complex to an analyte such as a ligand. Although in certain preferred embodiments, not all component subunits are associated with the membrane-associated subunit simultaneously.

A “multimeric multi-component membrane complex” contains more than one member for at least one type of subunits. The simplest multimeric multi-component membrane complex can be a receptor with two identical subunits (homodimer). A more complicated multimeric multi-component membrane complex may including two kinds of subunits (alpha and beta subunits), each type of subunits contains two members (α2β2). Alternatively, a multimeric multi-component membrane complex may include two alpha subunits, one beta subunit, and a gamma subunit, etc. According to the instant invention, only one of the two members in a homodimer may be a fusion protein with a fluorescent protein moiety. Alternatively, the two members of the same subunit can be fused to different fluorescent protein moieties (one alpha subunit fused to BFP, the other alpha subunit fused to GFP, etc.).

By “operably linked” is meant that a gene and transcriptional regulatory sequence(s) are connected in such a way as to permit expression of the gene in a manner dependent upon factors interacting with the regulatory sequence(s). In the case of the reporter gene, the DBD recognition element will also be operably linked to the reporter gene such that transcription of the reporter gene will be dependent, at least in part, upon bait-prey complexes bound to the recognition element.

The terms “pool” of polypeptides, “polypeptide library” or “combinatorial polypeptide library” are used interchangeably herein to indicate a variegated ensemble of polypeptide sequences, where the diversity of the library may result from cloning or be generated by mutagenesis. The terms “pool” of genes, “gene library” or “combinatorial gene library” have a similar meaning, indicating a variegated ensemble of nucleic acids.

By “protein” or “polypeptide” is meant a sequence of amino acids of any length, constituting all or a part of a naturally-occurring polypeptide or peptide, or constituting a non-naturally-occurring polypeptide or peptide (e.g., a randomly generated peptide sequence or one of an intentionally designed collection of peptide sequences).

“Quaternary structure” refers to complexes of 2 or more fully folded polypeptide chains held together by noncovalent forces but in precise ratios and with a precise 3-D configuration, i.e., the three-dimensional structure between subunits of a multi-component protein complex. Literally, quaternary is subsequent to tertiary (which refers to the three-dimensional structure of an entire polypeptide chain). In the context of biology, quaternary structures, such as enzymes, are assemblies of tertiary structural units, such as proteins. The assembly of bio-molecules into quaternary structures provides enhanced, multiple or novel functional roles. These assemblies may contain as few as two units, as in an enzyme complex, or hundreds, as in a virus. Often quaternary structure is organized symmetrically. This allows the formation of large complexes with only a few different tertiary units. In multiple unit (multimeric) complexes the single units (monomers) form contacts between each other. In some cases, but not always, the monomer must change its conformation (tertiary structure) in order to make these contacts. Insulin illustrates how the tertiary structure can be influenced by the quaternary organizational requirements. Enzymes exemplify the advantages of multimeric complexes that combine different functions. Multiple functionality is enhanced by forming multi-enzyme complexes. Hemoglobin is another example of quaternary structure that changes upon binding of oxygen by its individual subunits. Finally, large assemblies play not only functional but structural roles on the cellular level.

By “randomly generated” is meant sequences having no predetermined sequence; this is contrasted with “intentionally designed” sequences which have a DNA or protein sequence or motif determined prior to their synthesis.

As used herein, “recombinant cells” include any cells that have been modified by the introduction of heterologous DNA.

The terms “recombinant protein”, “heterologous protein” and “exogenous protein” are used interchangeably throughout the specification and refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid.

As used herein, a “reporter gene construct” is a nucleic acid that includes a “reporter gene” operatively linked to transcriptional regulatory sequences. Transcription of the reporter gene is controlled by these sequences. The activity of at least one or more of these control sequences is directly or indirectly regulated by a transcriptional complex recruited by virtue of interaction between the bait and prey fusion proteins. The transcriptional regulatory sequences can include a promoter and other regulatory regions that modulate the activity of the promoter, or regulatory sequences that modulate the activity or efficiency of the RNA polymerase that recognizes the promoter. Such sequences are herein collectively referred to as transcriptional regulatory elements or sequences. The reporter gene construct will also include a “DBD recognition element” which is a nucleotide sequence that is specifically bound by the DNA binding domain of the bait fusion protein. The DBD recognition element is located sufficiently proximal to the promoter sequence of the reporter gene so as to cause increased reporter gene expression upon recruitment of an RNA polymerase complex by a bait fusion protein bound at the recognition element.

As used herein, a “reporter gene” is a gene whose expression may be assayed; reporter genes may encode any protein that provides a phenotypic marker, for example: a protein that is necessary for cell growth or a toxic protein leading to cell death, e.g., a protein which confers antibiotic resistance or complements an auxotrophic phenotype; a protein detectable by a colorimetric/fluorometric assay leading to the presence or absence of color/fluorescence; or a protein providing a surface antigen for which specific antibodies/ligands are available.

By “altering the expression of the reporter gene” is meant a statistically significant increase or decrease in the expression of the reporter gene to the extent required for detection of a change in the assay being employed. It will be appreciated that the degree of change will vary depending upon the type of reporter gene construct or reporter gene expression assay being employed.

By “screening” is meant a process whereby a library of compound or genetic material encoding polypeptides is surveyed to determine whether there exists within this population one or more compounds/genes which possess a particular property, such as the ability to promote or inhibit FRET.

“Transmembrane protein” contains at least one stretch of polypeptide that spans a bio-membrane (transmembrane domain), such as the plasma membrane of a cell. There may be additionally intracellular or extracellular portions (or both) in the transmembrane protein. A transmembrane protein can also contain more than one transmembrane domains.

It is further noted that the following description of particular arrangements of polypeptide sequences in terms of being part of the fusion proteins is, in general, arbitrary. As will be apparent from the description, the polypeptide portions of any given components of fusion proteins may ordinarily be exchanged with one another. For example, the fluorescent protein moieties can be either at the N- or C-terminal or in the middle of a fusion protein. And it can be either intracellular or extracellular.

Each component of the system is now described in more detail.

III. Fusion Protein Constructs

One of the first steps in the use of the system of the present invention is to construct the donor fluorescent fusion protein and the acceptor fluorescent fusion protein. To do this, sequences encoding a protein or subunit of interest or a polypeptide library are cloned in-frame to a sequence encoding either a donor fluorescent moiety or an acceptor fluorescent protein moiety. Those skilled in the art will appreciate from the present disclosure that there are a wide variety of donor and acceptor fluorescent moieties that can be used to construct the said fusion proteins, including polypeptides derived from naturally occurring fluorescent proteins, as well as polypeptides derived from said natural fluorescent proteins artificially engineered to exhibit either enhanced fluorescent signals or shifted excitation and/or emission wave lengths.

A variety of Aequorea-related GFPs having useful excitation and emission spectra have been engineered by modifying the amino acid sequence of a naturally occurring GFP from Aequorea victoria. See, Prasher, D. C., et al., Gene, 111:229-233 (1992); Heim, R., et al., Proc. Natl. Acad. Sci., USA, 91:12501-04 (1994); U.S. Ser. No. 08/337,915, filed Nov. 10, 1994; International application PCT/US95/14692, filed Nov. 10, 1995; and U.S. Ser. No. 08/706,408, filed Aug. 30, 1996. The cDNA of GFP can be fused with those encoding many other proteins; the resulting fusions often are fluorescent and retain the biochemical features of the partner proteins. See, Cubitt, A. B., et al., Trends Biochem. Sci. 20:448-455 (1995). Mutagenesis studies have produced GFP mutants with shifted wavelengths of excitation or emission. See, Heim, R. & Tsien, R. Y. Current Biol. 6:178-182 (1996). Suitable pairs, for example a blue-shifted GFP mutant P4-3 (Y66H/Y 145F) and an improved green mutant S65T can respectively serve as a donor and an acceptor for fluorescence resonance energy transfer (FRET). See, Tsien, R. Y., et al., Trends Cell Biol. 3:242-245 (1993). A fluorescent protein is an Aequorea-related fluorescent protein if any contiguous sequence of 150 amino acids of the fluorescent protein has at least 85% sequence identity with an amino acid sequence, either contiguous or non-contiguous, from the wild type Aequorea green fluorescent protein. More preferably, a fluorescent protein is an Aequorea-related fluorescent protein if any contiguous sequence of 200 amino acids of the fluorescent protein has at least 95% sequence identity with an amino acid sequence, either contiguous or non-contiguous, from the wild type Aequorea green fluorescent protein. Similarly, the fluorescent protein can be related to Renilla or Phialidium wild-type fluorescent proteins using the same standards. Some Aequorea-related engineered versions described in Table I. Other variants or mutants are within the scope of the invention as described, for example, in the Examples.

TABLE I

| | | | | Extinc- | |
|---|---|---|---|---|---|
| | | | | tion | |
| | | | | Coeffi- | Quan- |
| | | Excitation | Emission | cient | tum |
| Clone | Mutation(s) | max(nm) | max(nm) | (M⁻¹cm⁻¹) | Yield |
| Wild | none | 395 (475) | 508 | 21,000 | 0.77 |
| type | | | | (7,150) | |
| P4 | Y66H | 383 | 447 | 13,500 | 0.21 |
| P4-3 | Y66H;Y14SF | 381 | 445 | 14,000 | 0.38 |
| W7 | Y66W;N146I | 433 (453) | 475 (501) | 18,000 | 0.67 |
| | | | | (17,100) | |
| | M153T | | | | |
| | V163A | | | | |
| | N212K | | | | |
| W2 | Y66W;1123V | 432 (453) | 480 | 10,000 | 0.72 |
| | | | | (9,600) | |
| | Y145H | | | | |
| | H14BR | | | | |
| | M153T | | | | |
| | V163A | | | | |
| | N212K | | | | |
| S65T | S65T | 489 | 511 | 39,200 | 0.68 |
| P4-1 | S65T;M153A | 504 (396) | 514 | 14,500 | 0.53 |
| | | | | (8,600) | |
| | K238E | | | | |
| S65A | S65A | 471 | 504 | | |
| S65C | S65C | 479 | 507 | | |
| S65L | S65L | 484 | 510 | | |
| Y66F | Y66F | 360 | 442 | | |
| Y66W | YE6W | 458 | 480 | | |
| 10c | 865G;V68L | 513 527 | | | |
| | S72A;T203Y | | | | |
| W1B | F64L;S65T | 432 (453) | 476 (503) | | |
| | Y66W;N146I | | | | |
| | M153T | | | | |
| | V163A | | | | |
| | N212K | | | | |
| Emerald | S6ST;S72A | 487 | 508 | | |
| | N149K | | | | |
| | M153T | | | | |
| | 1167T | | | | |
| Sapphire | S72A;Y14SF | 395 | 511 | | |
| | T203I | | | | |

Other fluorescent proteins can be used as the fluorescent moiety, such as, for example, yellow fluorescent protein from Vibrio fischeri strain Y-1, Peridinin-chlorophyll a binding protein from the dinoflagellate Symbiodinium sp. phycobiliproteins from marine cyanobacteria such as Synechococcus, e.g., phycoerythrin and phycocyanin, or oat phytochromes from oat reconstructed with phycoerythrobilin. These fluorescent proteins have been described in Baldwin, T. O., et al., Biochemistry 29:5509-5515 (1990), Morris, B. J., et al., Plant Molecular Biology, 24:673-677 (1994), and Wilbanks, S. M., et al., J. Biol. Chem. 268:1226-1235 (1993), and Li et al., Biochemistry 34:7923-7930 (1995).

The first test protein may be chosen from any protein of interest and includes proteins of unknown, known, or suspected diagnostic, therapeutic, or pharmacological importance. Exemplary proteins include, but are not limited to, oncoproteins (such as myc, particularly the C-terminus of myc, ras, src, fos, and particularly the oligomeric interaction domains of fos), tumor-suppressor proteins (such as p53, Rb, INK4 proteins [p16^{INK4a}, p15^{INK}^{4b}], CIP/KIP proteins [p21^{CIP1}, p₂₇^{KIP1}] or any other proteins involved in cell-cycle regulation (such as kinases and phosphatases). In other embodiments, the test polypeptide can be generated using all or a portion of a protein involved in signal transduction, including such motifs as SH2 and SH3 domains, ITAMs, ITIMs, kinase, phospholipase, or phosphatase domains, cytoplasmic tails of receptors and the like. Yet other preferred test fusion proteins are generated with cytoskeletal proteins or factors involved in transcription or translation, or portions thereof. Still other test fusion proteins can be generated with viral proteins.

In preferred embodiments, where the test protein includes a catalytic domain of an enzyme, the fusion protein is derived with a catalytically inactive mutant, most preferably a mutant which binds substrate with about the Kₘ of the wild-type enzyme but with a greatly diminished K_{cat} for the catalyzed reaction with the substrate. For example, mutation of a residue in the catalytic site of the enzyme can give rise to such catalytically inactive mutants. Particular examples include point mutation of the active site lysine of a kinase, the active site serine of a serine protease or the active site cysteine of a phosphatase. Thus, the binding of the first test polypeptide portion of the fusion protein to a polypeptide substrate presented by a second test fusion protein can be enhanced. In each case, the protein of interest is fused to donor or acceptor fluorescent moiety generally described herein.

In another preferred embodiment, the fluorescent protein moieties can be fused to different subunits of a multi-component membrane complex. In one embodiment, two different fluorescent moieties can be fused to two different kinds of subunits, such as the R1 chain and the R2 chain of the IFN-gamma receptor; in a related embodiment, two different fluorescent moieties can be fused to two different members of the same subunit (one to each member of a IFN-gamma R2 homodimer. In another related embodiment, only one R1 and one R2 is fused to fluorescent protein moieties, while the other R1 and R2 are not (for example, R1-BFP::R1::R2-GFP::R2). A skilled artisan should readily understand other numerous possible arrangement of fluorescent protein moieties in multi-component membrane complexes.

The use of recombinant DNA techniques to create a fusion gene, with the translational product being the desired test fusion proteins, is well known in the art. Essentially, the joining of various DNA fragments coding for different polypeptide sequences is performed in accordance with conventional techniques, employing blunt-ended or stagger-ended termini for ligation, restriction enzyme digestion to provide for appropriate termin, filling in of cohesive ends as appropriate, alkaline phosphatase treatment to avoid undesirable joining, and enzymatic ligation. Alternatively, the fusion gene can be synthesized by conventional techniques including automated DNA synthesizers. In another method, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, Eds. Ausubel et al. John Wiley & Sons: 1992).

It may be necessary in some instances to introduce an unstructured polypeptide linker region between the fluorescent moiety of the fusion protein and the test polypeptide sequence. Where the test fusion protein also includes oligomerization sequences, it may be preferable to situate the linker between the oligomerization sequences and the test polypeptide. The linker can facilitate enhanced flexibility of the fusion protein, and it can also reduce steric hindrance between the two fragments, and allow appropriate interaction between the two test polypeptide portions. The linker can also facilitate the appropriate folding of each fragment to occur. The linker can be of natural origin, such as a sequence determined to exist in random coil between two domains of a protein. An exemplary linker sequence is the linker found between the C-terminal and N-terminal domains of the RNA polymerase α subunit. Other examples of naturally occurring linkers include linkers found in the λcI and LexA proteins. Alternatively, the linker can be of synthetic origin. For instance, the sequence (Gly₄Ser)₃ can be used as a synthetic unstructured linker. Linkers of this type are described in Huston et al. (1988) PNAS 85:4879; and U.S. Pat. No. 5,091,513, both incorporated by reference herein. Another exemplary embodiment includes a poly alanine sequence, e.g., (Ala)₃.

In addition, some endogenous small molecules are naturally fluorescent, and can be used to label specific proteins. For example, many vitamins, co-enzymes, and nucleotides are naturally fluorescent. If these small molecules specifically bind to certain proteins, then these proteins can be excited at the excitation wavelength of these small molecules, without the need of generating a fusion protein with a fluorescent polypeptide. Most biological matter is fluorescent, albeit in the uv. Respective species include the three aromatic amino acids and proteins containing them, the green fluorescent protein (GFP), nucleic acids, flavine nucleotides and NADH, whilst NAD+ and most saccharides and lipids are non-fluorescent. In addition, most colored matter in nature including coumarins, flavones, anthocyans and chlorophylls (but with the notable exception of hem) is fluorescent. Fluorescent labels render a biomolecule (or a biological system) fluorescent so to make it amenable to fluorescence spectroscopy. Labels are preferably attached to the species of interest by covalent binding via a reactive group that forms a chemical bonds with other groups such as amino, hydroxy, sulfhydryl or carboxy. Labels are expected to be inert to other chemical species present in the environment, for example to pH. In order to reduce background luminescence of biological matter, labels preferably have long-wave excitation and emission, and/or long decay times so that background luminescence decays much faster than the luminescence of the label.

Numerous bioanalytical assays are based on the fact that NADH is fluorescent, while NAD⁺ is not. As a result, all enzymatic reactions based on NAD/NADH are amenable to fluorescence analysis, and this is widely exploited in practice, even though NADH has to be excited at around 350 nm which can cause substantial background fluorescence from other biomatter. On the other side, most assays are performed in the kinetic mode so that it is the relative signal change (above the background) that is measured rather than the total intensity (including the background) and this reduces interferences a lot.

The co-enzyme FAD is another strongly luminescent species but has found less wide applications because both the oxidized (FAD) and the reduced form (FADH₂) display fluorescence so that they are less useful for monitoring the course of a biochemical reaction. Their excitation is at around 450 nm, and fluorescence peaks at 512 nm. Both NADH and FAD have been shown to be useful for purposes of chemical sensing using immobilized reagents and, in some cases, using fiber optics waveguides.

Furthermore, proteins can be labeled in vitro via any of the art recognized means and then injected into test cells for the practice of the invention. For example, any fluorescent moieties (not necessarily fluorescent proteins) can be covalently attached to certain pre-selected proteins via chemical means, or non-covalently via strong biological binding affinities between certain molecules such as biotin and streptavidin. In the later case, a fluorescent moiety can be linked to streptavidin, while the protein of interest linked to biotin (or vice versa). Proteins labeled in such a way can then be used with a fusion protein containing a fluorescent protein moiety (such as GFP, BFP), or can be used with another similarly labeled protein with a different yet compatible fluorescent moiety for FRET. A skilled artisan will readily appreciate other variations of this embodiment without deviating from the sprit of the invention.

IV Host Cells

Host cells to be used can be any cell types, including both eukaryotic cells and prokaryotic cells. Preferably, they can be engineered to contain fluorescent moieties suitable for the assay. More preferably, the host cells include prokaryotic cells (bacteria), yeast, COS cell, HeLa cell, mammalian cells, established human cancer cell, cells isolated from patients with major human diseases such as cancer, diabetes, multiple sclerosis, etc. In addition, host cells should be able to express the appropriate genes at high levels.

The choice of appropriate host cell will also be influenced by the choice of detection signal and compatibility with the laser confocal microscope and FACS collector.

V. Microscope and Light Source

The excitation light source or microscope should be compatible for the purpose of performing assays on live cells, i.e., it should be able to overcome the tremendous amounts of light scattering, and thus artifacts, generated by live cells. Preferably, it is an instrument coupling a confocal microscope with a spectrofluorimeter. Fluorescence images are recorded on a confocal microscope based on substantial modifications done on a commercially available inverted microscope (Nikon, Diaphot 300, objective Nikon FLUOR 40, numerical aperture=1.3, oil immersion) without the laser that is usually supplied with the Nikon microscope. A laser beam from a separately housed titanium-sapphire femtosecond pulsed mode-lock tunable infrared laser was directed into the confocal microscope to excite the BFP or other donor fluorescent moieties. To measure FRET between donor and acceptor fluorescent moieties in a cell, the cell is excited with 2-photon excitation (half the energy of a one-photon excitation) light directly, with the cell itself capturing two-photons to obtain excitation at the usual excitation wavelength of the donor. In some experiments to examine the spectrum of the donor directly, the infrared laser emitting two-photon light is used with a doubling crystal to generate one-photon light to excite the donor, but this is not used for measuring FRET between donor and acceptor. For excitation of acceptor in a cell, a continuous wave argon laser was used with a band pass filter which generates the light capable of exciting the acceptor directly. The cells are excited by linearly polarized light through the back-port of the microscope.

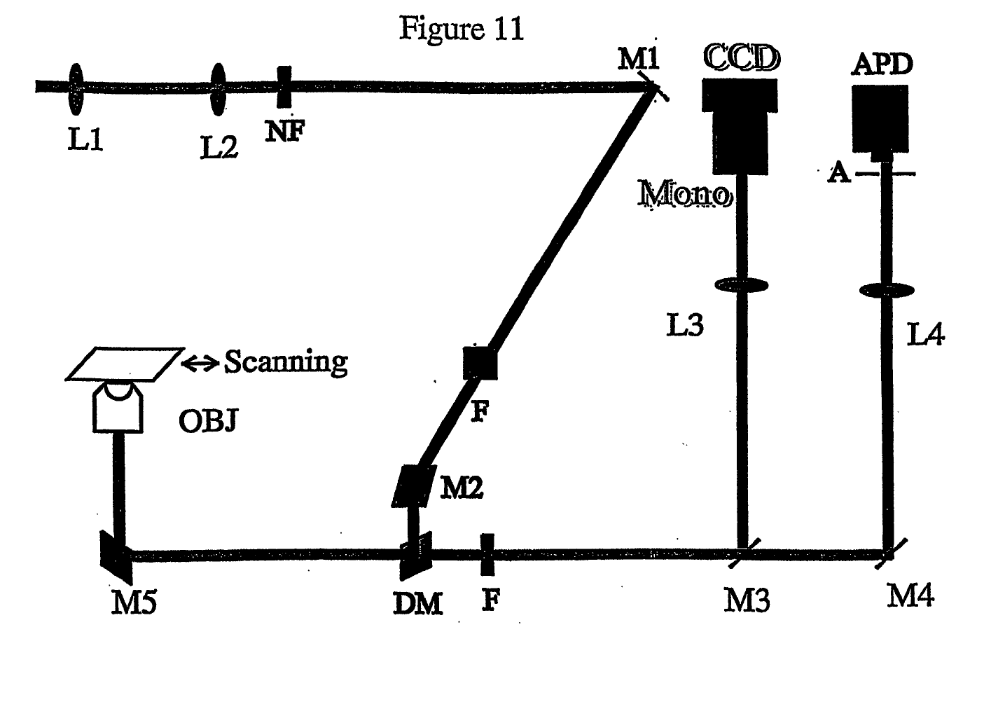

FIG. 11 illustrates a possible configuration of a microscope that can be used for the methods of the instant invention. FIG. 11 is a schematic of the confocal microscope coupled to lasers, photon counter, monochromater and CCD camera. Either single photon excitation at 488 nm of the GFP with an argon laser delivering 0.5 μW at the sample or a pulsed femtosecond mode-locked infrared Ti:sapphire laser (2 mW) tuned to 760 nm can be used. The lasers can be directly coupled to the microscope before the lens L1. The blue line represents the path of the light for excitation of the sample placed on the scanning platform. The green line represents the light path of the emission from the sample on the scanning platform back through the objective lens, to the mirror (M5), then passing through the dichromatic mirror (DM) along the path (green line) to the photon counting detector (APD) to collect the images or to the monochromoter (Mono) and spectral detector (CCD) to analyze the spectra. L1-L4 represent lenses; NF, a neutral density filter; M1-M5, mirrors; F, a band pass filter; DM is a dichromatic mirror; OBJ, the objective lens; APD, a photon counting detector; A, the aperture; CCD, the spectral detector; and Mono, the monochromater. The scanning of the sample can be done with a Princeton Instrument P-731 stage and controlled by a Digital instrument Nanoscope IIIA unit. M3 is a flipper mirror allowing the emission to reach either the CCD camera or the photon counting detector (APD).

The cells can be mounted on a closed loop scan unit (Queensgate, Ascot, U.K., S222, scanning area 35 μm×35 μm) controlled by a modified Nanoscope E controller (Digital Instruments, Santa Barbara, Calif.), and the emitted fluorescence is collected by a single photon-counting module (EG&G, Salem, Mass., SPCM-AQ-161) for fluorescence images or by a combination of a monochromator (Acton Research, Acton, Mass., model 150) and a backilluminated nitrogen cooled charge-coupled device camera (Princeton Instruments, Trenton, N.J.) for spectra. Fluorescence images are obtained in the same way as described previously (Bopp M A, Jia Y, Li L, Cogdell R J, Hochstrasser R M. “Fluorescence and photobleaching dynamics of single light-harvesting complexes,” Proc Natl Acad Sci U S A. 94:10630-10635, 1997; Bopp M A, Sytnik A, Howard T D, Cogdell R J, Hochstrasser R M. “The dynamics of structural deformations of immobilized single light-harvesting complexes,” Proc Natl Acad Sci U S A. 96:11271-11276, 1999).

For high-throughput screening, a standard flow cytometer can be modified with the lasers described above to analyze the FRET. For detecting the FRET between donor and acceptor fluorescent moieties, cells will be excited with two-photon excitation light from an infrared laser so that the two photon capture by the cells will yield and effective one-photon excitation and emission at the wavelength of the acceptor. In addition, other methods can be used for two-photon excitation in live cells that was used to obtain 3-D images (Bahlmann K, Jakobs S, Hell SW “4Pi-confocal microscopy of live cells,” Ultramicroscopy 87:155-164, 2001). The isolated cells then can be analyzed individually by PCR or other amplification techniques.

The method is also applicable to FACS units such as the Meridian instrument that have been developed to scan and sort cells on monolayers rather than in a flowing fluid.

It should also be understood that although confocal laser source is preferably used for excitation of single cells, many other laser sources may also be applicable under certain conditions, such as when populations of cells rather than single cells are used under low background conditions. It should also be understood that the wavelengths mentioned in this application are for illustrative purpose only, and is by no means limiting. With the discovery of future fluorescent molecules/proteins with unique excitation and emission wavelengths, these wavelengths can also be properly used to practice the instant invention.

VI. Multi-Component Membrane Complexes

In one embodiment, the method of the present invention can be used for identifying effectors or ligands of a receptor protein or complex thereof. In general, the method is characterized by the use of a test cell which includes a target receptor or ion channel protein whose signal transduction activity can be modulated by interaction with an extracellular signal, the transduction activity being able to generate a detectable signal.

In general, such embodiments of the subject method are characterized by the use of a mixture of cells expressing a target receptor protein or ion channel capable of transducing a detectable FRET signal in the reagent cell. The receptor/channel protein can be either endogenous or heterologous. In combination with the disclosed detection means, a culture of the instant reagent cells will provide means for detecting agonists or antagonists of receptor function.

The ability of particular peptides or ligands to modulate a signal transduction activity of the target receptor or channel can be scored for by detecting up or down-regulation of the detection signal. For example, the appearance or disappearance of the FRET signal as the result of ligand binding. Alternatively, second messenger generation (e.g. GTPase activity, phospholipid hydrolysis, or protein phosphorylation patterns as examples) can be measured directly. Alternatively, the use of an indicator gene can provide a convenient readout. In other embodiments a detection means consists of an indicator gene. In any event, a statistically significant change in the detection signal can be used to facilitate identification of compounds which modulate receptor or ion channel activities.

By this method, peptides/ligands which induce a signal pathway from a particular receptor or channel can be identified. If a test peptide does not appear to induce the activity of the receptor/channel protein, the assay may be repeated as described above, and modified by the introduction of a step in which the reagent cell is first contacted with a known activator of the target receptor/channel to induce signal transduction, and the test peptide can be assayed for its ability to inhibit the activated receptor/channel, e.g., to identify antagonists. In yet other embodiments, peptides can be screened for those which potentiate the response to a known activator of the receptor.

With respect to the receptor or ion channel, it may be endogenously expressed by the host cell, or it may be expressed from a heterologous gene that has been introduced into the cell. Methods for introducing heterologous DNA into eukaryotic cells are of course well known in the art and any such method may be used. In addition, DNA encoding various receptor proteins is known to those of skill in the art or it may be cloned by any method known to those of skill in the art. In certain embodiments, such as when an exogenous receptor is expressed, it may be desirable to inactivate, such as by deletion, a homologous receptor present in the cell.

In particular, the assays can be used to test functional ligand-receptor or ligand-ion channel interactions for cell surface-localized receptors and channels. As described in more detail below, the subject assay can be used to identify effectors of multi-component membrane complexes, for example, G protein-coupled receptors, receptor tyrosine kinases, cytokine receptors, and ion channels. In certain embodiments the method described herein is used for identifying ligands for “orphan receptors” for which no ligand is known.

In preferred embodiments, the receptor is a cell surface receptor, such as: a receptor tyrosine kinase, e.g., an EPH receptor; an ion channel; a cytokine receptor;

an multi-subunit immune recognition receptor, a chemokine receptor; a growth factor receptor, or a G-protein coupled receptor, such as a chemoattracttractant peptide receptor, a neuropeptide receptor, a light receptor, a neurotransmitter receptor, or a polypeptide hormone receptor.

Preferred G protein coupled receptors include α1A-adrenergic receptor, α1B-adrenergic receptor, β2-adrenergic receptor, α2B-adrenergic receptor, β1-adrenergic receptor, β2-adrenergic receptor, β3-adrenergic receptor, ml acetylcholine receptor (AChR), m2 AChR, m3 AChR, m4 AChR, m5 AChR, D1 dopamine receptor, D2 dopamine receptor, D3 dopamine receptor, D4 dopamine receptor, D5 dopamine receptor, A1 adenosine receptor, A2b adenosine receptor, 5-HT1a receptor, 5-HT1b receptor, 5HT1-like receptor, 5-HT1d receptor, 5HT1d-like receptor, 5HT1d beta receptor, substance K (neurokinin A) receptor, fMLP receptor, fMLP-like receptor, angiotensin II type 1 receptor, endothelin ETA receptor, endothelin ETB receptor, thrombin receptor, growth hormone-releasing hormone (GHRH) receptor, vasoactive intestinal peptide receptor, oxytocin receptor, somatostatin SSTR1 and SSTR2, SSTR3, cannabinoid receptor, follicle stimulating hormone (FSH) receptor, leutropin (LH/HCG) receptor, thyroid stimulating hormone (TSH) receptor, thromboxane A2 receptor, platelet-activating factor (PAF) receptor, C5a anaphylatoxin receptor, Interleukin 8 (IL-8) 1L-8RA, IL-8RB, Delta Opioid receptor, Kappa Opioid receptor, mip-1/RANTES receptor, Rhodopsin, Red opsin, Green opsin, Blue opsin, metabotropic glutamate mGluR1-6, histamine H2 receptor, ATP receptor, neuropeptide Y receptor, amyloid protein precursor receptor, insulin-like growth factor II receptor, bradykinin receptor, gonadotropin-releasing hormone receptor, cholecystokinin receptor, melanocyte stimulating hormone receptor, antidiuretic hormone receptor, glucagon receptor, and adrenocorticotropic hormone II receptor.

Preferred EPH receptors include eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk and nuk receptors.

A. Cytokine Receptors

In one embodiment the target receptor is a cytokine receptor. Cytokines are a family of soluble mediators of cell-to-cell communication that includes interleukins, interferons, and colony-stimulating factors. The characteristic features of cytokines lie in their functional redundancy and pleiotropy. Most of the cytokine receptors that constitute distinct superfamilies do not possess intrinsic protein tyrosine kinase domains, yet receptor stimulation usually invokes rapid tyrosine phosphorylation of intracellular proteins, including the receptors themselves. Many members of the cytokine receptor superfamily activate the Jak protein tyrosine kinase family, with resultant phosphorylation of the STAT transcriptional activator factors. IL-2, IL-7, IL-2 and Interferon γ have all been shown to activate Jak kinases (Frank et al (1995) Proc Natl Acad Sci USA 92:7779-7783); Scharfe et al. (1995) Blood 86:2077-2085); (Bacon et al. (1995) Proc Natl Acad Sci USA 92:7307-7311); and (Sakatsume et al (1995) J. Biol Chem 270:17528-17534). Events downstream of Jak phosphorylation have also been elucidated. For example, exposure of T lymphocytes to IL-2 has been shown to lead to the phosphorylation of signal transducers and activators of transcription (STAT) proteins STAT1α, STAT2α, and STAT3, as well as of two STAT-related proteins, p94 and p95. The STAT proteins were found to translocate to the nucleus and to bind to a specific DNA sequence, thus suggesting a mechanism by which IL-2 may activate specific genes involved in immune cell function (Frank et al. supra). Jak3 is associated with the gamma chain of the IL-2, IL-4, and IL-7 cytokine receptors (Fujii et al. (1995) Proc Natl Acad Sci 92:5482-5486) and (Musso et al (1995) J. Exp Med. 181:1425-1431). The Jak kinases have also been shown to be activated by numerous ligands that signal via cytokine receptors such as, growth hormone and erythropoietin and IL-6 (Kishimoto (1994) Stem cells Suppl 12:37-44).

B. Multisubunit Immune Recognition Receptor (MIRR)

In another embodiment the receptor is a multisubunit receptor. Receptors can be comprised of multiple proteins referred to as subunits, one category of which is referred to as a multisubunit receptor is a multisubunit immune recognition receptor (MIRR). MIRRs include receptors having multiple noncovalently associated subunits and are capable of interacting with src-family tyrosine kinases. MIRRs can include, but are not limited to, B cell antigen receptors, T cell antigen receptors, Fc receptors and CD22. One example of an MIRR is an antigen receptor on the surface of a B cell. To further illustrate, the MIRR on the surface of a B cell comprises membrane-bound immunoglobulin (mlg) associated with the subunits Ig-α and Ig-β or Ig-γ, which forms a complex capable of regulating B cell function when bound by antigen. An antigen receptor can be functionally linked to an amplifier molecule in a manner such that the amplifier molecule is capable of regulating gene transcription.

Src-family tyrosine kinases are enzymes capable of phosphorylating tyrosine residues of a target molecule. Typically, a src-family tyrosine kinase contains one or more binding domains and a kinase domain. A binding domain of a src-family tyrosine kinase is capable of binding to a target molecule and a kinase domain is capable of phosphorylating a target molecule bound to the kinase. Members of the src family of tyrosine kinases are characterized by an N-terminal unique region followed by three regions that contain different degrees of homology among all the members of the family. These three regions are referred to as src homology region 1 (SH1), src homology region 2 (SH2) and src homology region 3 (SH3). Both the SH2 and SH3 domains are believed to have protein association functions important for the formation of signal transduction complexes. The amino acid sequence of an N-terminal unique region, varies between each src-family tyrosine kinase. An N-terminal unique region can be at least about the first 40 amino acid residues of the N-terminal of a src-family tyrosine kinase.

Syk-family kinases are enzymes capable of phosphorylating tyrosine residues of a target molecule. Typically, a syk-family kinase contains one or more binding domains and a kinase domain. A binding domain of a syk-family tyrosine kinase is capable of binding to a target molecule and a kinase domain is capable of phosphorylating a target molecule bound to the kinase. Members of the syk-family of tyrosine kinases are characterized by two SH2 domains for protein association function and a tyrosine kinase domain.

A primary target molecule is capable of further extending a signal transduction pathway by modifying a second messenger molecule. Primary target molecules can include, but are not limited to, phosphatidylinositol 3-kinase (PI-3K), P21 rasGAPase-activating protein and associated P190 and P62 protein, phospholipases such as PLCγ1 and PLCγ2, MAP kinase, Shc and VAV. A primary target molecule is capable of producing second messenger molecule which is capable of further amplifying a transduced signal. Second messenger molecules include, but are not limited to diacylglycerol and inositol 1,4,5-triphosphate (IP3). Second messenger molecules are capable of initiating physiological events which can lead to alterations in gene transcription. For example, production of IP3 can result in release of intracellular calcium, which can then lead to activation of calmodulin kinase 11, which can then lead to serine phosphorylation of a DNA binding protein referred to as ets-1 protoonco-protein. Diacylglycerol is capable of activating the signal transduction protein, protein kinase C which affects the activity of the API DNA binding protein complex. Signal transduction pathways can lead to transcriptional activation of genes such as c-fos, egr-1, and c-myc.

Shc can be thought of as an adaptor molecule. An adaptor molecule comprises a protein that enables two other proteins to form a complex (e.g., a three molecule complex). Shc protein enables a complex to form which includes Grb2 and SOS. Shc comprises an SH2 domain that is capable of associating with the SH2 domain of Grb2.

Molecules of a signal transduction pathway can associate with one another using recognition sequences. Recognition sequences enable specific binding between two molecules. Recognition sequences can vary depending upon the structure of the molecules that are associating with one another. A molecule can have one or more recognition sequences, and as such can associate with one or more different molecules.

Signal transduction pathways for MIRR complexes are capable of regulating the biological functions of a cell. Such functions can include, but are not limited to the ability of a cell to grow, to differentiate and to secrete cellular products. MIRR-induced signal transduction pathways can regulate the biological functions of specific types of cells involved in particular responses by an animal, such as immune responses, inflammatory responses and allergic responses. Cells involved in an immune response can include, for example, B cells, T cells, macrophages, dendritic cells, natural killer cells and plasma cells. Cells involved in inflammatory responses can include, for example, basophils, mast cells, eosinophils, neutrophils and macrophages. Cells involved in allergic responses can include, for example mast cells, basophils, B cells, T cells and macrophages.

In exemplary embodiments of the subject assay, the detection signal is a second messengers, such as a phosphorylated src-like protein, includes reporter constructs or indicator genes which include transcriptional regulatory elements such as serum response element (SRE), 12-O-tetradecanoyl-phorbol-13-acetate response element, cyclic AMP response element, c-fos promoter, or a CREB-responsive element.

C. Receptor Tyrosine Kinases

In still another embodiment, the target receptor is a receptor tyrosine kinase. The receptor tyrosine kinases can be divided into five subgroups on the basis of structural similarities in their extracellular domains and the organization of the tyrosine kinase catalytic region in their cytoplasmic domains. Sub-groups I (epidermal growth factor (EGF) receptor-like), II (insulin receptor-like) and the eph/eck family contain cysteine-rich sequences (Hirai et al., (1987) Science 238:1717-1720 and Lindberg and Hunter, (1990) Mol. Cell. Biol. 10:6316-6324). The functional domains of the kinase region of these three classes of receptor tyrosine kinases are encoded as a contiguous sequence (Hanks et al. (1988) Science 241:42-52). Subgroups III (platelet-derived growth factor (PDGF) receptor-like) and IV (the fibro-blast growth factor (FGF) receptors) are characterized as having immunoglobulin (Ig)-like folds in their extracellular domains, as well as having their kinase domains divided in two parts by a variable stretch of unrelated amino acids (Yanden and Ullrich (1988) supra and Hanks et al. (1988) supra).

The family with by far the largest number of known members is the EPH family. Since the description of the prototype, the EPH receptor (Hirai et al. (1987) Science 238:1717-1720), sequences have been reported for at least ten members of this family, not counting apparently orthologous receptors found in more than one species. Additional partial sequences, and the rate at which new members are still being reported, suggest the family is even larger (Maisonpierre et al. (1993) Oncogene 8:3277-3288; Andres et al. (1994) Oncogene 9:1461-1467; Henkemeyer et al. (1994) Oncogene 9:1001-1014; Ruiz et al. (1994) Mech Dev 46:87-100; Xu et al. (1994) Development 120:287-299; Zhou et al. (1994) J Neurosci Res 37:129-143; and references in Tuzi and Gullick (1994) Br J Cancer 69:417-421). Remarkably, despite the large number of members in the EPH family, all of these molecules were identified as orphan receptors without known ligands.

The expression patterns determined for some of the EPH family receptors have implied important roles for these molecules in early vertebrate development. In particular, the timing and pattern of expression of sek, mek4 and some of the other receptors during the phase of gastrulation and early organogenesis has suggested functions for these receptors in the important cellular interactions involved in patterning the embryo at this stage (Gilardi-Hebenstreit et al. (1992) Oncogene 7:2499-2506; Nieto et al. (1992) Development 116:1137-1150; Henkemeyer et al., supra; Ruiz et al., supra; and Xu et al., supra). Sek, for example, shows a notable early expression in the two areas of the mouse embryo that show obvious segmentation, namely the somites in the mesoderm and the rhombomeres of the hindbrain; hence the name sek, for segmentally expressed kinase (Gilardi-Hebenstreit et al., supra; Nieto et al., supra). As in Drosophila, these segmental structures of the mammalian embryo are implicated as important elements in establishing the body plan. The observation that Sek expression precedes the appearance of morphological segmentation suggests a role for sek in forming these segmental structures, or in determining segment-specific cell properties such as lineage compartmentation (Nieto et al., supra). Moreover, EPH receptors have been implicated, by their pattern of expression, in the development and maintenance of nearly every tissue in the embryonic and adult body. For instance, EPH receptors have been detected throughout the nervous system, the testes, the cartilaginous model of the skeleton, tooth primordia, the infundibular component of the pituitary, various epithelia tissues, lung, pancreas, liver and kidney tissues. Observations such as this have been indicative of important and unique roles for EPH family kinases in development and physiology, but further progress in understanding their action has been severely limited by the lack of information on their ligands.

As used herein, the terms “EPH receptor” or “EPH-type receptor” refer to a class of receptor tyrosine kinases, comprising at least eleven paralogous genes, though many more orthologs exist within this class, e.g. homologs from different species. EPH receptors, in general, are a discrete group of receptors related by homology and easily recognizable, e.g., they are typically characterized by an extracellular domain containing a characteristic spacing of cysteine residues near the N-terminus and two fibronectin type III repeats (Hirai et al. (1987) Science 238:1717-1720; Lindberg et al. (1990) Mol Cell Biol 10:6316-6324; Chan et al. (1991) Oncogene 6:1057-1061; Maisonpierre et al. (1993) Oncogene 8:3277-3288; Andres et al. (1994) Oncogene 9:1461-1467; Henkemeyer et al. (1994) Oncogene 9:1001-1014; Ruiz et al. (1994) Mech Dev 46:87-100; Xu et al. (1994) Development 120:287-299; Zhou et al. (1994) J Neurosci Res 37:129-143; and references in Tuzi and Gullick (1994) Br. J. Cancer 69:417-421). Exemplary EPH receptors include the eph, elk, eck, sek, mek4, hek, hek2, eek, erk, tyro1, tyro4, tyro5, tyro6, tyro11, cek4, cek5, cek6, cek7, cek8, cek9, cek10, bsk, rtk1, rtk2, rtk3, myk1, myk2, ehk1, ehk2, pagliaccio, htk, erk and nuk receptors. The term “EPH receptor” refers to the membrane form of the receptor protein, as well as soluble extracellular fragments which retain the ability to bind the ligand of the present invention.

D. G Protein-Coupled Receptors

One family of signal transduction cascades found in eukaryotic cells utilizes heterotrimeric “G proteins.” Many different G proteins are known to interact with receptors. G protein signaling systems include three components: the receptor itself, a GTP-binding protein (G protein), and an intracellular target protein.

The cell membrane acts as a switchboard. Messages arriving through different receptors can produce a single effect if the receptors act on the same type of G protein. On the other hand, signals activating a single receptor can produce more than one effect if the receptor acts on different kinds of G proteins, or if the G proteins can act on different effectors.

In their resting state, the G proteins, which consist of alpha (α), beta (β) and gamma (γ) subunits, are complexed with the nucleotide guanosine diphosphate (GDP) and are in contact with receptors. When a hormone or other first messenger binds to receptor, the receptor changes conformation and this alters its interaction with the G protein. This spurs the α subunit to release GDP, and the more abundant nucleotide guanosine triphosphate (GTP), replaces it, activating the G protein. The G protein then dissociates to separate the α subunit from the still complexed beta and gamma subunits. Either the Gα subunit, or the Gβγ complex, depending on the pathway, interacts with an effector. The effector (which is often an enzyme) in turn converts an inactive precursor molecule into an active “second messenger,” which may diffuse through the cytoplasm, triggering a metabolic cascade. After a few seconds, the Gα converts the GTP to GDP, thereby inactivating itself. The inactivated Gα may then reassociate with the Gβγ complex.

Hundreds, if not thousands, of receptors convey messages through heterotrimeric G proteins, of which at least 17 distinct forms have been isolated. Although the greatest variability has been seen in the a subunit, several different b and g structures have been reported. There are, additionally, several different G protein-dependent effectors.

Most G protein-coupled receptors are comprised of a single protein chain that is threaded through the plasma membrane seven times. Such receptors are often referred to as seven-transmembrane receptors (STRs). More than a hundred different STRs have been found, including many distinct receptors that bind the same ligand, and there are likely many more STRs awaiting discovery.

In addition, STRs have been identified for which the natural ligands are unknown; these receptors are termed “orphan” G protein-coupled receptors, as described above. Examples include receptors cloned by Neote et al. (1993) Cell 72, 415; Kouba et al. FEBS Lett. (1993) 321, 173; Birkenbach et al.(1993) J. Virol. 67, 2209.

The “exogenous receptors” of the present invention may be any G protein-coupled receptor which is exogenous to the cell which is to be genetically engineered for the purpose of the present invention. This receptor may be a plant or animal cell receptor. Screening for binding to plant cell receptors may be useful in the development of, e.g., herbicides. In the case of an animal receptor, it may be of invertebrate or vertebrate origin. If an invertebrate receptor, an insect receptor is preferred, and would facilitate development of insecticides. The receptor may also be a vertebrate, more preferably a mammalian, still more preferably a human, receptor. The exogenous receptor is also preferably a seven transmembrane segment receptor.

Known ligands for G protein coupled receptors include: purines and nucleotides, such as adenosine, cAMP, ATP, UTP, ADP, melatonin and the like; biogenic amines (and related natural ligands), such as 5-hydroxytryptamine, acetylcholine, dopamine, adrenaline, adrenaline, adrenaline., histamine, noradrenaline, noradrenaline, noradrenaline., tyramine/octopamine and other related compounds; peptides such as adrenocorticotrophic hormone (acth), melanocyte stimulating hormone (msh), melanocortins, neurotensin (nt), bombesin and related peptides, endothelins, cholecystokinin, gastrin, neurokinin b (nk3), invertebrate tachykinin-like peptides, substance k (nk2), substance p (nk1), neuropeptide y (npy), thyrotropin releasing-factor (trf), bradykinin, angiotensin ii, beta-endorphin, c5a anaphalatoxin, calcitonin, chemokines (also called intercrines), corticotrophic releasing factor (crf), dynorphin, endorphin, fmlp and other formylated peptides, follitropin (fsh), fungal mating pheremones, galanin, gastric inhibitory polypeptide receptor (gip), glucagon-like peptides (glps), glucagon, gonadotropin releasing hormone (gnrh), growth hormone releasing hormone(ghrh), insect diuretic hormone, interleukin-8, leutropin (lh/hcg), met-enkephalin, opioid peptides, oxytocin, parathyroid hormone (pth) and pthrp, pituitary adenylyl cyclase activiating peptide (pacap), secretin, somatostatin, thrombin, thyrotropin (tsh), vasoactive intestinal peptide (vip), vasopressin, vasotocin; eicosanoids such as ip-prostacyclin, β-prostaglandins, α-thromboxanes; retinal based compounds such as vertebrate 11-cis retinal, invertebrate 11-cis retinal and other related compounds; lipids and lipid-based compounds such as cannabinoids, anandamide, lysophosphatidic acid, platelet activating factor, leukotrienes and the like; excitatory amino acids and ions such as calcium ions and glutamate.

Suitable examples of G-protein coupled receptors include, but are not limited to, dopaminergic, muscarinic cholinergic, α-adrenergic, β-adrenergic, opioid (including delta and mu), cannabinoid, serotoninergic, and GABAergic receptors. Preferred receptors include the 5HT family of receptors, dopamine receptors,C5a receptor and FPRL-1 receptor, cyclo-histidyl-proline-diketoplperazine receptors, melanocyte stimulating hormone release inhibiting factor receptor, and receptors for neurotensin, thyrotropin releasing hormone, calcitonin, cholecytokinin-A, neurokinin-2, histamine-3, cannabinoid, melanocortin, or adrenomodulin, neuropeptide-Y1 or galanin. Other suitable receptors are listed in the art. The term “receptor,” as used herein, encompasses both naturally occurring and mutant receptors.

Many of these G protein-coupled receptors, like the yeast a- and α-factor receptors, contain seven hydrophobic amino acid-rich regions which are assumed to lie within the plasma membrane. Specific human G protein-coupled STRs for which genes have been isolated and for which expression vectors could be constructed include those listed herein and others known in the art. Thus, the gene would be operably linked to a promoter functional in the cell to be engineered and to a signal sequence that also functions in the cell. For example in the case of yeast, suitable promoters include Ste2, Ste3 and gal10. Suitable signal sequences include those of Ste2, Ste3 and of other genes which encode proteins secreted by yeast cells. Preferably, when a yeast cell is used, the codons of the gene would be optimized for expression in yeast. See Hoekema et al.,(1987) Mol. Cell. Biol., 7:2914-24; Sharp, et al., (1986)14:5125-43.

The homology of STRs is discussed in Dohlman et al., Ann. Rev. Biochem., (1991) 60:653-88. When STRs are compared, a distinct spatial pattern of homology is discernible. The transmembrane domains are often the most similar, whereas the N- and C-terminal regions, and the cytoplasmic loop connecting transmembrane segments V and VI are more divergent.

The functional significance of different STR regions has been studied by introducing point mutations (both substitutions and deletions) and by constructing chimeras of different but related STRs. Synthetic peptides corresponding to individual segments have also been tested for activity. Affinity labeling has been used to identify ligand binding sites.

It is conceivable that when the host cell is a yeast cell, a foreign receptor will fail to functionally integrate into the yeast membrane, and there interact with the endogenous yeast G protein. More likely, either the receptor will need to be modified (e.g., by replacing its V-V1 loop with that of the yeast STE2 or STE3 receptor), or a compatible G protein should be provided.

If the wild-type exogenous G protein-coupled receptor cannot be made functional in yeast, it may be mutated for this purpose. A comparison would be made of the amino acid sequences of the exogenous receptor and of the yeast receptors, and regions of high and low homology identified. Trial mutations would then be made to distinguish regions involved in ligand or G protein binding, from those necessary for functional integration in the membrane. The exogenous receptor would then be mutated in the latter region to more closely resemble the yeast receptor, until functional integration was achieved. If this were insufficient to achieve functionality, mutations would next be made in the regions involved in G protein binding. Mutations would be made in regions involved in ligand binding only as a last resort, and then an effort would be made to preserve ligand binding by making conservative substitutions whenever possible.

Preferably, the yeast genome is modified so that it is unable to produce the yeast receptors which are homologous to the exogenous receptors in functional form. Otherwise, a positive assay score might reflect the ability of a peptide to activate the endogenous G protein-coupled receptor, and not the receptor of interest.

(i) Chemoattractant Receptors

The N-formyl peptide receptor is a classic example of a calcium mobilizing G protein-coupled receptor expressed by neutrophils and other phagocytic cells of the mammalian immune system (Snyderman et al. (1988) In Inflammation: Basic Principles and Clinical Correlates, pp.309-323). N-formyl peptides of bacterial origin bind to the receptor and engage a complex activation program that results in directed cell movement, release of inflammatory granule contents, and activation of a latent NADPH oxidase which is important for the production of metabolites of molecular oxygen. This pathway initiated by receptor-ligand interaction is critical in host protection from pyogenic infections. Similar signal transduction occurs in response to the inflammatory peptides C5a and IL-8.

Two other formyl peptide receptor like (FPRL) genes have been cloned based on their ability to hybridize to a fragment of the NFPR cDNA coding sequence. These have been named FPRL1 (Murphy et al. (1992) J. Biol. Chem. 267:7637-7643) and FPRL2 (Ye et al. (1992) Biochem Biophys Res. Comm. 184:582-589). FPRL2 was found to mediate calcium mobilization in mouse fibroblasts transfected with the gene and exposed to formyl peptide. In contrast, although FPRL1 was found to be 69% identical in amino acid sequence to NFPR, it did not bind prototype N-formyl peptides ligands when expressed in heterologous cell types. This lead to the hypothesis of the existence of an as yet unidentified ligand for the FPRLI orphan receptor (Murphy et al. supra).

(ii) G Proteins

In the case of an exogenous G-protein coupled receptor, the yeast cell must be able to produce a G protein which is activated by the exogenous receptor, and which can in turn activate the yeast effector(s). The art suggests that the endogenous yeast Gα subunit (e.g., GPA) will be often be sufficiently homologous to the “cognate” Gα subunit which is natively associated with the exogenous receptor for coupling to occur. More likely, it will be necessary to genetically engineer the yeast cell to produce a foreign Ga subunit which can properly interact with the exogenous receptor. For example, the Ga subunit of the yeast G protein may be replaced by the Gα subunit natively associated with the exogenous receptor.

Dietzel and Kurjan, (1987) Cell, 50:1001) demonstrated that rat Gas functionally coupled to the yeast Gβγ complex. However, rat Gαi2 complemented only when substantially overexpressed, while GaO did not complement at all. Kang, et al., Mol. Cell. Biol., (1990)10:2582). Consequently, with some foreign Gα subunits, it is not feasible to simply replace the yeast Ga.

If the exogenous G protein coupled receptor is not adequately coupled to yeast Gβγ by the Gα subunit natively associated with the receptor, the Gα subunit may be modified to improve coupling. These modifications often will take the form of mutations which increase the resemblance of the Gα subunit to the yeast Gα while decreasing its resemblance to the receptor-associated Gα. For example, a residue may be changed so as to become identical to the corresponding yeast Gα residue, or to at least belong to the same exchange group of that residue. After modification, the modified Gα subunit might or might not be “substantially homologous” to the foreign and/or the yeast Gα subunit.

The modifications are preferably concentrated in regions of the Gα which are likely to be involved in Gβγ binding. In some embodiments, the modifications will take the form of replacing one or more segments of the receptor-associated Gα with the corresponding yeast Gα segment(s), thereby forming a chimeric Gα subunit. (For the purpose of the appended claims, the term “segment” refers to three or more consecutive amino acids.) In other embodiments, point mutations may be sufficient.

This chimeric Got subunit will interact with the exogenous receptor and the yeast Gβγ complex, thereby permitting signal transduction. While use of the endogenous yeast Gβγ is preferred, if a foreign or chimeric Gβγ is capable of transducing the signal to the yeast effector, it may be used instead.

VII. Pharmaceutical Preparations of Identified Agents

After identifying certain test peptides/ligands in the subject assay, e.g. as potential surrogate ligands, or receptor antagonists, the practitioner of the subject assay will continue to test the efficacy and specificity of the selected peptides both in vitro and in vivo. Whether for subsequent in vivo testing, or for administration to an animal as an approved drug, peptides identified in the subject assay, or peptidomimetics thereof, can be formulated in pharmaceutical preparations for in vivo administration to an animal, preferably a human.

The peptides selected in the subject assay, or a pharmaceutically acceptable salt thereof, may accordingly be formulated for administration with a biologically acceptable medium, such as water, buffered saline, polyol (for example, glycerol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the compound, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations”. Based on the above, such pharmaceutical formulations include, although not exclusively, solutions or freeze-dried powders of the compound in association with one or more pharmaceutically acceptable vehicles or diluents, and contained in buffered media at a suitable pH and isosmotic with physiological fluids. In preferred embodiment, the peptide can be disposed in a sterile preparation for topical and/or systemic administration. In the case of freeze-dried preparations, supporting excipients such as, but not exclusively, mannitol or glycine may be used and appropriate buffered solutions of the desired volume will be provided so as to obtain adequate isotonic buffered solutions of the desired pH. Similar solutions may also be used for the pharmaceutical compositions of compounds in isotonic solutions of the desired volume and include, but not exclusively, the use of buffered saline solutions with phosphate or citrate at suitable concentrations so as to obtain at all times isotonic pharmaceutical preparations of the desired pH, (for example, neutral pH).

VIII. Exemplary Uses of the Invention

The present invention can be used, inter alia, for identifying protein-protein interactions, e.g., for generating protein linkage maps, for detecting quaternary structural changes, for identifying therapeutic targets, and/or for general cloning strategies. As described above, the system can be used in conjunction with a cDNA library to produce a variegated array of test proteins which can be screened for interaction with, for example, a known protein expressed as the corresponding fusion protein. In other embodiments, both test proteins can be derived to each provide variegated libraries of polypeptide sequences. One or both libraries can be generated by random or semi-random mutagenesis. For example, random libraries of polypeptide sequences can be “crossed” with one another by simultaneous expression in the subject assay. Such embodiments can be used to identify novel binding pairs of polypeptides.

Alternatively, the invention can be used to map residues of a protein involved in a known protein-protein interaction. Thus, for example, various forms of mutagenesis can be utilized to generate a combinatorial library of either test polypeptides, and the ability of the corresponding fusion protein to generate FRET signal can be assayed. Mutations which result in diminished (or potentiated) binding between the test fusion proteins can be detected by the strength/existence/absence of the FRET signal. For example, mutants of a particular protein which alter interaction of that protein with another protein can be generated and isolated from a library created, for example, by alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of a protein, e.g., to establish binding domains.

In other embodiments, the method can be designed for the isolation of genes encoding proteins which physically interact with a protein/drug complex. The method relies on detecting the FRET signal in the presence of the drug, such as rapamycin, FK506 or cyclosporin. If the test fusion proteins are able to interact in a drug-dependent manner, the interaction may be detected by FRET signal.

Another aspect of the present invention relates to the use of the method in the development of assays which can be used to screen for drugs which are either agonists or antagonists of a protein-protein interaction of therapeutic consequence. In a general sense, the assay evaluates the ability of a compound to modulate binding between two given test polypeptides. In a preferred embodiment, the method evaluates the ability of a compound to induce quaternary structual changes in a multi-component membrane complex. Exemplary compounds which can be screened include peptides, nucleic acids, carbohydrates, small organic molecules, and natural product extract libraries, such as isolated from animals, plants, fungus and/or microbes.

In many drug screening programs which test libraries of compounds and natural extracts, high throughput assays are desirable in order to maximize the number of compounds surveyed in a given period of time. The screening assay described above can be carried out in such a format, and accordingly may be used as a “primary” screen. Accordingly, in an exemplary screening assay of the present invention, the method can be used to promote or disrupt certain interactions between two known proteins, either with or without the presence of certain physiological stimuli. In that respect, a first test protein is covalently linked to a donor fluorescent protein moiety, and a second test protein is covalently linked to an acceptor fluorescent protein moiety. If the two test proteins/subunits constitutively interact with each other in the live host cells under physiological conditions, FRET may be observed after activation of the donor fluorescent protein moiety by a laser beam of appropriate wave length. A large number of pharmaceutical preparations can be quickly tested on these FRET-signal bearing cells in an automated HTS so that anything specifically diminishing the FRET signal can be readily identified. Alternatively, pharmaceutical preparations specifically promoting certain interactions between two known proteins can also be identified. These process can be done with or without the presence of certain natural/physiological stimulations (such as growth factors), so that pharmaceutical preparations capable of overcoming or bypassing these stimulations can be identified.

In another exemplary embodiment, a therapeutic target devised as the FRET pair complex is contacted with a peptide library with the goal of identifying peptides which potentiate or inhibit certain interactions or certain quaternary conformations.

Many techniques are known in the art for expression peptide libraries. In one embodiment, the peptide library is provided as part of a chimeric thioredoxin protein, e.g., expressed as part of the active loop.

Moreover, it will be apparent that the subject FRET-based assay can be used generally to detect mutations in other cellular proteins which disrupt protein-protein interactions. For example, it has been shown that the transcription factor E2F-4 is bound to the p130 pocket protein, and that such binding effectively suppresses E2F-4-mediated trans-activation required for control of G₀/G₁ transition. Mutants which result in disruption of this interaction can be detected in the subject assay.

Similarly, Rb and Rb-like proteins (such as p107) act to control cell-cycle progression through the formation of complexes with several cellular proteins. In fact, a recent article concerning familial retinoblastoma has reported a new class of Rb mutants found in retinal lesions, which mutants were defective in protein binding (“pocket”) activity (see, for example, Kratzke et al. (1994) Oncogene 9:1321-1326). Moreover, mutant forms of c-myc have been demonstrated in various lymphomas, e.g., Burkitt lymphomas, which mutants are resistant to p107-mediated suppression. Accordingly, the diagnostic assay of the present invention can be used to detect mutations in Rb or Rb-like proteins which disrupt binding to other cellular proteins, e.g., myc, E2F, c-Ab1, or upstream binding factor (UBF), or vice-versa.

In another embodiment, the subject diagnostic assay can be employed to detect mutations which disrupt binding of the p53 protein with other cellular proteins, as for example, the Wilm's tumor suppresser protein WT1. Recent observations by Maheswaran et al. (1993, PNAS 90:5100-5104) have demonstrated that p53 can physically interact with WT1, and that this interaction modulates the ability of each protein to transactivate their respective targets. In fact, in contrast to the proposed function of WT1 as a transcriptional repressor, potent transcriptional activation by WT1 of reporter genes driven by EGR1 in cells lacking wild type p53 indicates that transcriptional repression is not an intrinsic property of WT1. Instead, transcriptional repression by WT1 may result from its interaction with p53. Accordingly, mutations in p53 which do not effect the cellular concentration of this protein, but which rather down regulate its ability to bind to and repress WT1, may give rise to Wilm's tumors, and other disease states associated with deregulation of WT1.

In still another embodiment, the diagnostic FRET assay can be used to detect mutations in pairs of signal transduction proteins. For example, the present assay can be used to detect mutations in the ras protein or other cellular proteins which interact with ras, e.g., ras GTPase activating proteins (GAPs).

Another aspect of the invention provides a method to develop/screen for new antibiotics directed against bacteria, fungi, parasites, or other pathogens, etc. The methods of the instant invention can be used to identify and/or screen for natural or synthesized compounds that specifically or preferentially target (disrupt function of) organelle or multi-component complexes of these pathogens. For example, Azithromycin is an important antibiotic for the treatment of several different Gram-positive and Gram-negative bacterial infections. Erythromycin and clarithromycin are less useful antibiotics against Gram-negative infections. These antibiotics specifically target the synthesis and assembly of 50S prokaryotic ribosomal large subunit (but not eukaryotic ribosomal subunits or the 30S prokaryotic ribosomal large subunit). Therefore, if two known 50S ribosomal subunit component proteins are labeled with fluorescent moieties, FRET signal can result if these two component proteins are within close proximity to each other. Disruption of the 50S ribosomal subunit by a test compound will reduce the FRET signal, thus identifying the test compounds as a useful antibiotics. Chemical modifications can be used to modify the structure of the lead compound identified above, and further screening can be carried out to optimize the inhibitory effects.

Yet another aspect of the invention provides a method for determination of all possible interactions of all proteins in a given proteome. In one respect, a library of all proteins within a given genome can be introduced into a host cell population, in the form of fusion proteins with a certain donor fluorescent protein, by way of overproduction in the host cells using certain mammalian expression techniques. The number of individual cDNA expressed in any given cell can be controlled so that each cell, on average, expresses one distinct cDNA. A second library of all proteins can then be introduced into the said host cell population, in the form of fusion proteins with a certain acceptor fluorescent protein. Interactions between proteins belonging to the said two groups, with or without stimuli, will generate FRET signals which can be used to sort out cells containing such signals using a FACS machine. Genes within each collected single cell can then be identified by techniques well-known in the art such as single cell PCR. Only about 50 bases needs to be sequenced to identify the gene. However, the entire gene can be sequenced to ascertain whether it is the full-length gene or one of the splice variants, etc. By doing this with each human protein, the entire proteome map can be obtained. This can be applied to mammalian cells, microorganisms or any cells including both eukaryotic and prokaryotic cells.

Alternatively, a first library of all proteins within a given genome can be introduced into a host cell population, in the form of fusion proteins with a certain donor fluorescent protein, by way of overproduction in the host cells using certain mammalian expression techniques. Any given protein of interest can then be produced in this library of cells as a fusion protein with an acceptor fluorescent moiety. Interactions between the protein of interest and any protein belong to the said library, with or without stimuli, will generate FRET signals which can be used to sort out cells containing such signals using a FACS machine. Genes within each collected single cell can then be identified by techniques well-known in the art such as single cell PCR. By testing each and every individual protein within a given proteome, it is possible to construct a detailed protein-protein interaction map within that proteome.

In another embodiment of the invention, 10-100 different fluorescent fusion proteins can be simultaneously introduced into the cells. Fewer cells would then need to be screened to obtain positive cells exhibiting FRET signal. However, each cDNA identified from such positive cells will then need to be tested separately. This, however, could be especially useful when different fluorescent tags with different wavelengths are available to allow a matrix of fluorescent interactions.

In another embodiment of the invention, the method provides a way to compare normal and diseased cells. By introducing the cDNA library into diseased cells, it will be possible to examine the differences in the proteome map for various cancers, autoimmune diseases, cardiovascular diseases, genetic diseases, etc; for environmental changes and insults; or for infectious diseases. This will provide not only insights into how these disease states or environmental changes affect the interaction of difference proteins in live cells, but also new insight into pathways affected in these diseases. This will be especially important for multifactorial diseases where genetics is very cumbersome and takes years, if not decades to sort out.

In another embodiment of the invention, the method can be applied to microorganisms, where it would be useful to identify differences between the proteome map in pathogenic vs. nonpathogenic variants so that new antibiotics and other agents to kill microorganisms and various parasites can be developed. Exemplification The invention, now being generally described, will be more readily understood by reference to the following examples, which are included merely for purposes of illustration of certain aspects and embodiments of the present invention and are not intended to limit the invention.

### EXAMPLE 1

### FRET Between IFN-γ Receptors

As mentioned before, when two proteins interact, the distance should be sufficiently short (less than 10 nm, preferably less than 5 nm, or even 1 nm) that if one were tagged with a donor fluorescent moiety and the other were tagged with an acceptor fluorescent protein moiety, the fluorescent markers will be very close together. If two fluorescent molecules are sufficiently close to each other, photon energy can transfer from a donor molecule to an acceptor molecule, a phenomenon known as fluorescent resonance energy transfer (FRET) which has been used to quantitatively analyze protein-protein interactions in vitro. To our knowledge, this has never been attempted in an intact cell with cytokine or other receptors.

Interferon-gamma (IFN-γ) is a protein secreted by lymphocytes, specifically T-cells and NK-cells, that plays a prominent role in the activation of the immune system. Therefore, it is of significant interest to understand the IFN-γ receptor and the mechanism of its signal transduction. The IFN-γ receptor complex is composed of at least four transmembrane proteins: two molecules of the IFN-γ binding chain, IFN-γR1, and two molecules of the accessory receptor chain, IFN-γR2. When IFN-γ binds to the receptor complex, receptor-associated kinases, members of the Jak kinase family, are activated, which in turn phosphorylate transcription factors called Stat proteins. The dogma has been that the receptor complex is assembled during its activation from disassembled receptor components. However, there is no direct evidence about the structure of the receptor complex in its resting or activated states, or of the nature of interactions that occur among the four receptor chains in either state.

A mammalian expression vector was used to express human interferon γ receptor chains one and two (IFN-γR1 and IFN-γR2) with blue- and green-fluorescent proteins (BFP and GFP) fused to the carboxyl terminus of the chains, respectively. A tandem vector was used so that both chains were expressed in COS-1 cells under the control of the EF-1 promoter. A single cell expressing both of these chains was used to carry out the experiment to examine the effect of human IFN-γ on the distance between the chains and to determine if the chains were associated prior to addition of ligand. This is accomplished by using FRET.

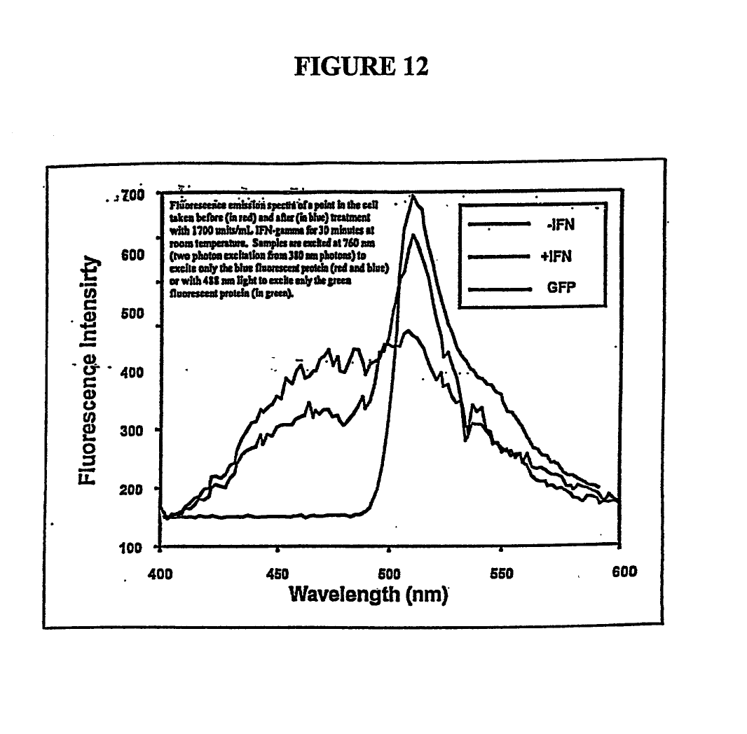

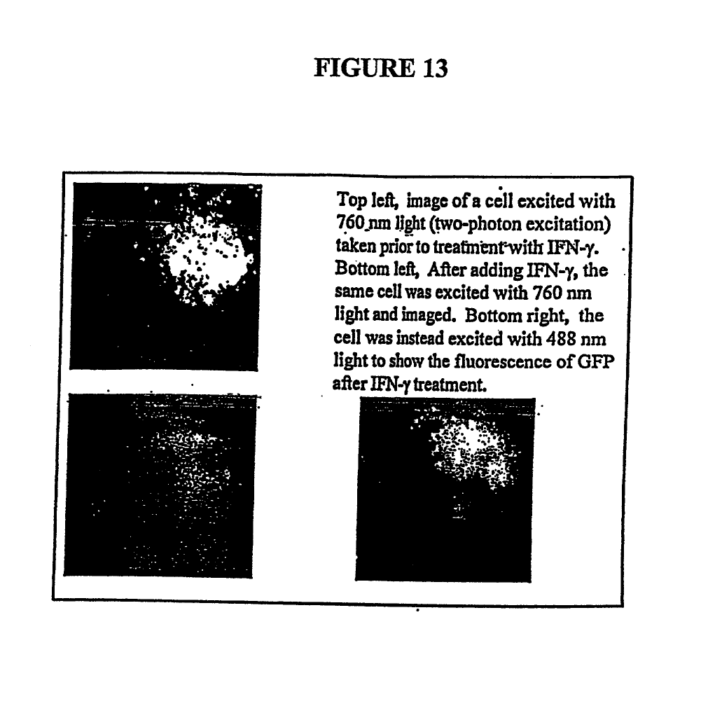

It was found that there was a FRET signal between the fluorescent receptor pairs in the absence of IFN-γ treatment. However, the efficiency of FRET changed significantly after treatment of cells with IFN-γ so that the FRET signal is almost completely abolished (FIGS. 12 and 13). In the meantime, the fluorescent signal of GFP excited at its excitation wavelength was shown to illustrate the emission spectrum. (FIG. 12). These data show that these receptor chains are constitutively associated with each other prior to the addition of their ligand; and addition of the ligand increases the distance between the intracellular domains of these chains.

Based on this observation, it is obvious that this technology can be used to screen for compounds that bind to and influence ligand-receptor interactions in intact cells with the hope of finding pharmacological targets of receptors.

### EXAMPLE 2

### Preassociation and Ligand-Induced Changes of the Interferon Gamma Receptor Complex in Cells Introduction

Specific protein-protein interactions are fundamental to many cellular functions. There interactions are involved in formation of functional replication, transcription, splicing and translation complexes, signal transduction pathways, cytoskeletal organization (e.g., microtubule polymerization), receptor-ligand binding, organization of multi-subunit enzyme complexes, and most cell functions.

Investigation of protein-protein interactions under physiological conditions has been problematic. Considerable effort has been made to identify proteins that bind to proteins of interest. Typically, these interactions have been detected by using co-precipitation experiments in which an antibody to a known protein is mixed with a cell extract and used to precipitate the known protein and any proteins which are stably associated with it. However, the method does not yield data in real time in cells. Another approach has been to use an interaction trap system (the yeast two-hybrid system) to identify polypeptide sequences which bind to a predetermined polypeptide sequence present in a fusion protein in cells (1). As originally developed, the yeast two-hybrid system requires that both proteins to be tested must be in the nucleus for the transcriptional activation to occur. To overcome this limitation, a method that does not involve transcription was developed to identify proteins that interact in the cytoplasm (2). Typically, the two-hybrid method is used to identify novel polypeptide sequences which interact with a known protein (3-10). However, the two-hybrid systems do not permit real time measurements of the interactions in cells.

Measurement of protein-protein interactions in vivo by non-invasive techniques can help to validate the physiological significance of the interactions and can also aid in identifying changes that occur in a cell or organism in response to physiological stimuli. One way to accomplish this is through fluorescent resonance energy transfer (FRET). FRET is a powerful spectroscopic technique that has been used to determine protein:protein interactions, and distances between two proteins in complexes. About 25 years ago (11) we used FRET and fluorescence lifetime measurements to determine the distance between two regions of the ribosome. Because FRET is a real-time technique, changes in protein:protein interactions or protein positions in complexes can be determined over the course of time or modulation of the system being analyzed. Because very little is known about the physical association of specific receptor chains of multichain complexes and whether chains from one receptor type directly interact with another (12), the use of FRET to determine this should provide an answer these questions by applying the technique to cells.

FRET has begun to be used to determine interactions of proteins on the surface of cells. Monoclonal antibodies (mAbs) to cell surface receptors or other protein were labeled chemically with fluorescein isothiocyanate (FITC) and Cy3 dyes. The in vitro fluorescent-labeled mAbs directed against cell surface receptors were able to measure changes in the distance of the receptor components as a function of ligand concentrations (13-16). The cloning of green fluorescent protein (GFP) has allowed biologists to label proteins with the fluorescent GFP using genetic engineering (17, 18). The pituitary transcription factor (Pit-1) fused to BFP (BFP-Pit-1) was confirmed to interact with the protein c-Est-1 fused to GFP (GFP-Ets-1) by FRET, an association that was previously shown by co-immunoprecipitation (19). The association of Bcl-2 and Beclin fused to various mutants of GFP was determined by FRET (20). Additional reports in the last few years have begun to determine association of proteins in cells with the use of GFP and BFP or cyan fluorescent protein (CFP) and yellow fluorescent protein (YFP) pairs (21-33). The use of fluorescence lifetime imaging microscopy (FLIM) can eliminate the need for spectral separation and problems in FRET (34) as we found previously by pure spectral methods (11). Another technique, fluorescence recovery after photobleaching (FRAP), was used to demonstrate the interaction of Ras and RapI in cells (35).

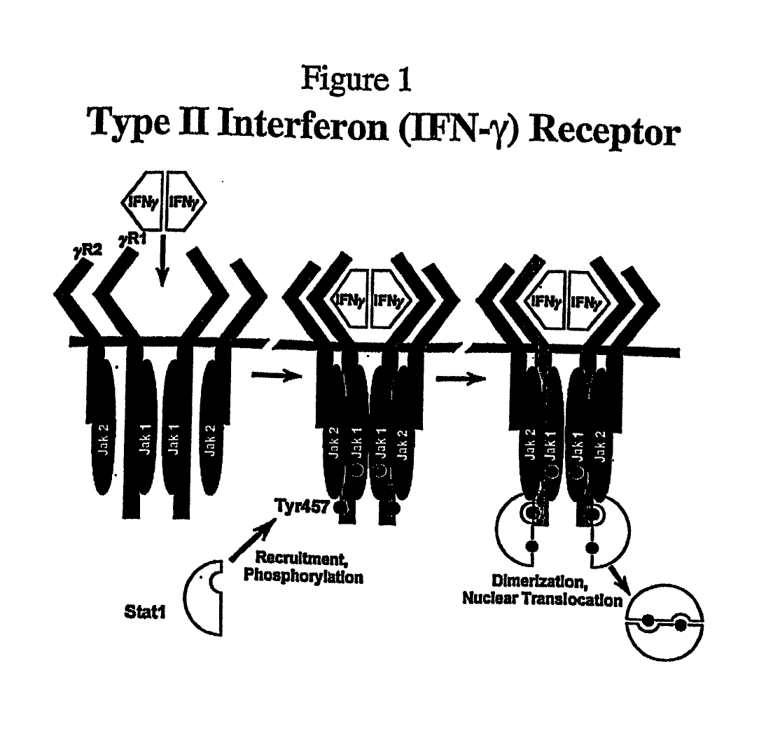

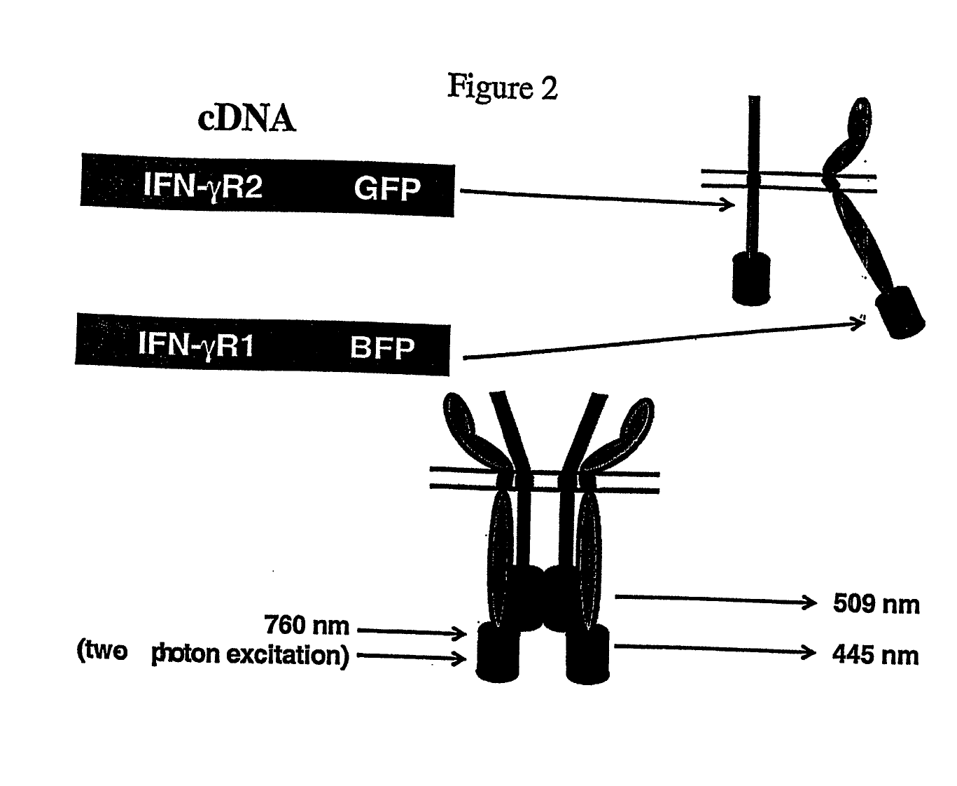

Interferons initiate signal transduction through specific cell surface receptors (12, 36-38). Interferon gamma (IFN-γ) binds to the IFN-γ receptor binding subunit (IFN-γR1; receptor chain one), a species-specific cell-surface receptor chain (39, 40). A second transmembrane protein (IFN-γR2) (41-43) is required (FIG. 1). Both 1FN-γR1 and IFN-γR2 chains are transmembrane proteins. The overall objective of our efforts is to provide an improved method to detect protein-protein interactions under physiological conditions in live cells by using FRET and to determine effects of ligands and other agents on the interactions by examining the IFN-Y receptor complex in detail by FRET. The results will have many useful applications in understanding receptor structure and provide a basis for high throughput screening of receptor ligands in single living cells.

Although a great deal of information has accumulated about cell surface receptors from biochemical observations, no direct measurements of receptor structure have been made in cells in the presence and absence of ligand. In most cases, the models of multi chain receptor complexes were deduced from a combination of cross-linking, immunoprecipitation of the constituent proteins and delineation of the signal transduction events. For example, based on a wide variety of studies it has been considered dogma that ligands join surface receptor chains that are not associated prior to attachment of ligand (44-50). All these results were deduced from indirect measurements. However, some more recent observations have begun to suggest that receptor chains can be preassociated in the cell membrane (51-76). To provide a direct way to clarify the various models of receptor action, we established an approach to measure interactions of receptor chains in living cells. To assess the structure of the receptor chains and the effects of ligands directly, we used fluorescence resonance energy transfer (FRET) and focused on the interferon gamma (IFN-γ) receptor. The chains and functions of the interferon gamma (IFN-γ) receptor complex have been defined in some detail over the past two decades (12, 37, 77-79). Indirect evidence exists about the structure of the interferon-gamma (IFN-γ) receptor complex prior to its activation by IFN-γ. Most investigators have concluded that the IFN-γ receptor and other receptors are assembled after ligand binding because only then was optimal immunoprecipitation of both chains obtained with an antibody to chain one (80-82). Accordingly, most models of the IFN-γ receptor consider it to consist of four chains: two IFN-γR1 and two IFN-γR2 chains consistent with a large body of data. After binding of ligand, it was concluded that the chains of the IFN-γ receptor complex are brought together and signal transduction initiated by ligand binding (FIG. 1). Here we report the results of FRET in examining receptor structure directly and provide evidence for a new model of receptor structure and function.

Materials and Methods

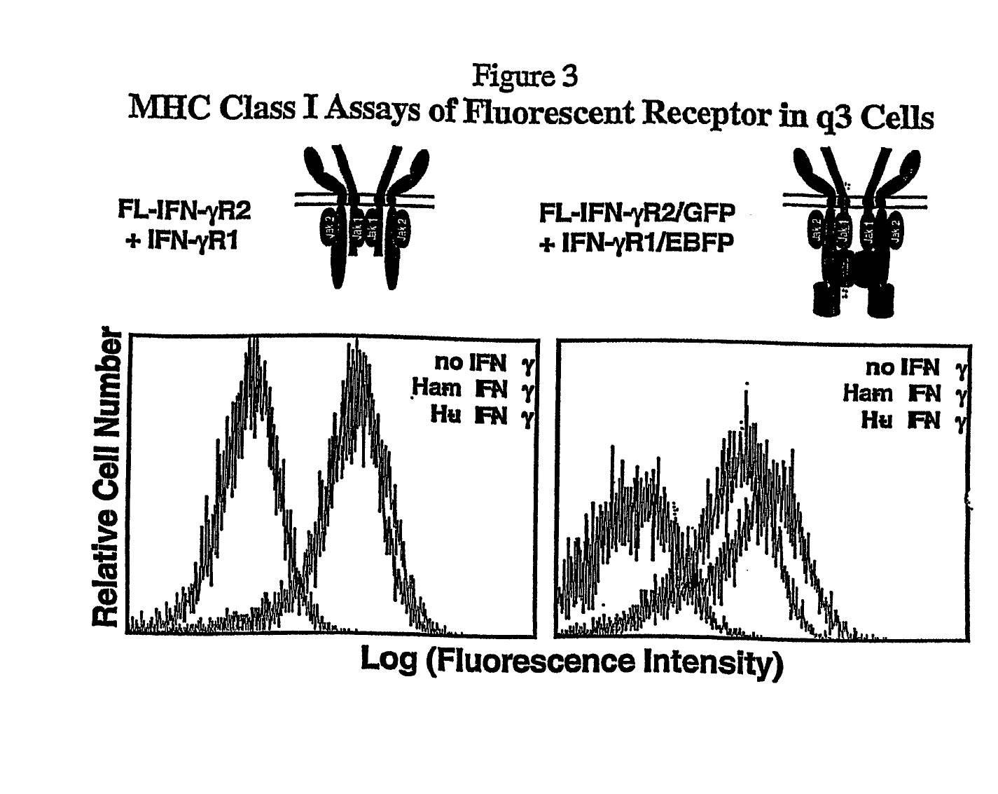

Construction of Vectors to Measure FRET in Cells and Transfections. As noted above, green and blue fluorescent proteins (GFP and BFP) were discovered and developed by a number of groups that enabled these fluorescent protein to be seen and localized in cells; and several groups have begun to use FRET to study the interactions of proteins in cells. In our studies, the IFN-γ receptor complex (FIG. 1) was used as the model system to evaluate the ability of FRET to provide insight into the structure of the receptor and to determine the molecular dynamics after attachment of the ligand IFN-γ to the receptor. In order to carry out these studies, IFN-γR1 and IFN-γR2 chains with BFP (or EBFP) and GFP fused to the carboxyl termini of their intracellular domains were constructed (FIG. 2, top). By fusing the DNA encoding the blue fluorescent protein (BFP) to the DNA encoding the carboxyl terminus of the IFN-γR1 chain and by fusing the DNA encoding GFP to the DNA encoding the carboxyl terminus of the IFN-γR2 chain, the cells produce IFN-γR11/BFP and IFN-γR2/GFP. In this way, the BFP and GFP were fused to the intracellular domains of IFN-γR1 and IFN-γR2, respectively (FIG. 2), to monitor interactions among these two receptor chains in intact cells-with and-without IFN-γ treatment. In all-constructions, the enhanced BFP (EBFP) with mammalian codons optimized was used unless otherwise specified; and all IFN-γR2 constructions contained the flag (FL) epitope, but this is not noted on the figures for simplicity, but it is noted in the legends to the figures. The detailed construction of FL-IFN-γR2/yR2 (first yR2 represents extracellular domain; second yR2, intracellular domain) was described in detail elsewhere (83, 84). It differs from the wild-type IFN-γR2 sequence in the following ways: the FLAG epitope was placed between the end of the putative signal peptide sequence and the putative beginning of the extracellular domain, and an NheI site was engineered into the beginning of the transmembrane domain, producing a three-amino acid mutation. Neither change affected the function of IFN-γR2. Because it was necessary have cells express two proteins labeled with EBFP and GFP at similar levels, we used a single vector expressing both proteins for transfection rather than co-transfection with two vectors. Thus, tandem vectors, in which transcription of each cDNA is controlled by its own separate promoter and polyadenylation signal on a single plasmid, were synthesized as described (84). The plasmid harboring the IFN-γR1 chain (or the IL-10R1 chain) was digested at the 3′ end either NruI or MluI, and at the 5′ end with either BglII, AatII or PvuI. The large fragment, retaining the IFN-γR1 cDNA and its expression elements was retained. The plasmid harboring the FL-IFN-γR2 chain (or the FL-IL-10R2 chain) was digested with either BglII, AatII or PvuI at its 5′ end and either SmaI or BssHII at its 3′ end. The larger fragment, retaining the FL-IFN-γR2 cDNA and its expression elements, was retained. Ligation of the cohesive ends yielded tandem vectors. To prepare EBFP or GFP fusion products, the segments encoding EBFP or GFP were amplified by PCR from appropriate vectors (85, 86) and then fused to the vectors expressing FL-IFN-γR2, IFN-γR1 or IL-10 with EBFP or GFP attached to the 3′ terminus of the expression construct to make FL-IFN-γR2/EBFP, FL-IFN-γR2/GFP, IFN-γR1/EBFP and IL-10/GFP, respectively. Schematic illustrations of the resultant EBFP and GFP labeled receptor chains are shown in FIG. 2 and FIG. 3. Transient transfections in COS-1 cells by the DEAE-dextran protocol (87) were used for all experiments except where noted.

Stable transformants were made in the CHO-derived cell line q3 by the Lipofectin method and limiting dilution clonal isolation.

Analysis of FRET Between Receptor Chains in Single Cells

Fret and equipment used. To demonstrate GFP and BFP fluorescence and FRET, a confocal microscope was modified to include a monochrometer associated with a back illumination liquid nitrogen cooled CCD camera so that fluorescence emission spectra could be obtained from illuminated cells. The S65T variant of GFP with an excitation maximum at 488 nm was used in all our studies (88). The enhanced GFP (EGFP) optimized for mammalian codons has the same excitation and emission maxima as GFP (S65T), 488 and 509 nm, respectively. We used GFP rather than EGFP in these experiments. Single photon excitation at 488 nm of the GFP with an argon laser delivering 0.5 μW at the sample yielded the signature GFP emission having a maximum at 509 nm. The BFP and EBFP have excitation and emission maxima at 380 nm and 445 nm. Because we found that excitation at 380 of cells produced very high background fluorescence, we used two-photon excitation to substantially reduce the sample excitation volume along with quartz cover slides, which resulted in a significantly decreased background fluorescence. The infrared light produced little or no cellular damage compared to ultraviolet light. To excite the BFP at its excitation maximum of 380 nm, a pulsed femtosecond mode-locked infrared Ti:sapphire laser (2 mW) was tuned to 760 nm. As illustrated in the lower part of FIG. 2, two-photon excitation (760 nm) of BFP (or EBFP) effectively excites the protein at it is maximum absorption at 380 nm to produce an emission spectrum with a maximum at 445 nm. If FRET occurs between BFP and GFP, then the emission maximum of GFP at 509 nm will be observed. We used both BFP and EBFP as noted in the figures.

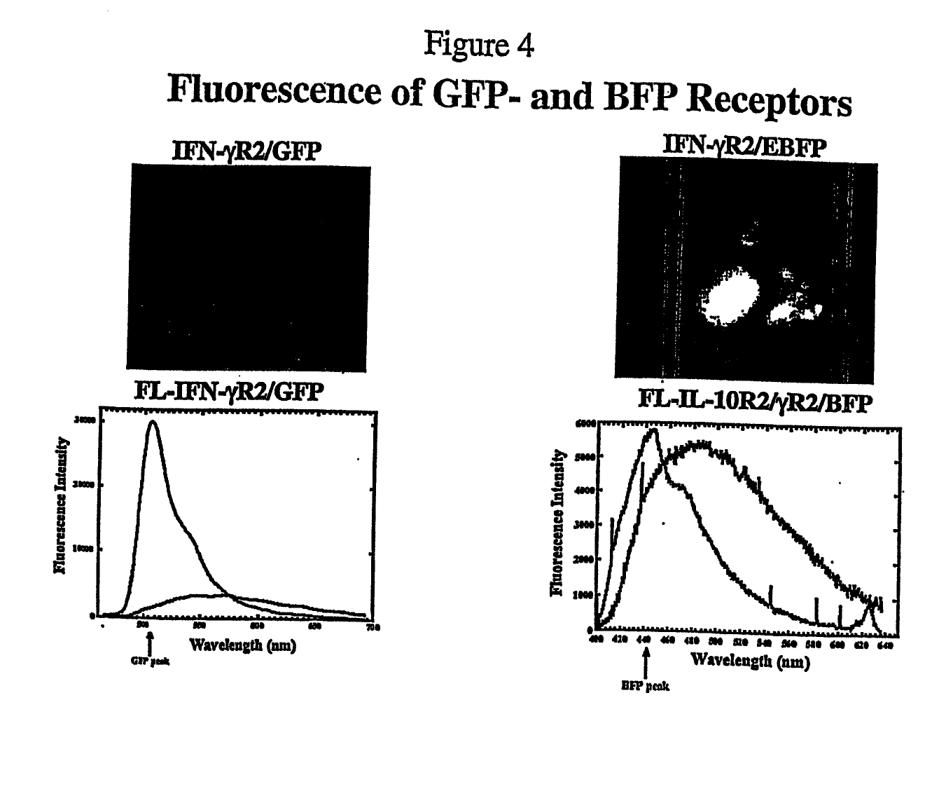

Images, Spectra and Activity of human IFN-γR2/GFP and IFN-γR1/BFP transfected into COS-1 cells. The constructs expressing IFN-γR2/GFP and IFN-γR2/EBFP were individually transfected into COS-1 cells and images taken with a camera attached to a confocal microscope. The images show that both IFN-γR2/GFP (FIG. 4, top right) and IFN-γR2/EBFP (enhanced BFP; FIG. 4, bottom right) were visualized by epifluorescence after transfection after exciting the IFN-γR2/GFP at 488 and the IFN-γR2/EBFP at 380 nm. Similar images were obtained for IFN-γR1/EGFP (not shown). To demonstrate that each of the constructs was functional, a tandem vector coexpressing IFN-γR2/GFP and IFN-γR1/EBFP was transfected into Chinese hamster ovary cells (CHO) expressing the complementary human chain (42, 78, 87). MHC class I induction by IFN-γ demonstrated that each chain was functional (data not shown). The MHC Class I surface antigen induction in response to IFN-γ in CHO q3 cells expressing IFN-γR2/GFP and IFN-γR1/BFP (FIG. 3, right panels) show that both receptor chains are functional. Similar activity measurements with other receptor chains such as IFN-γR2/EGFP, IFN-γR2/EBFP and FL-IL-1 OR2/GFP demonstrated that all these receptor chains with fused fluorescent proteins at their carboxy termini were functional. The spectral signature of GFP and BFP was seen in cells expressing IFN-γR2/GFP (FIG. 4, left panels; excited at 488 nm) and IL-10R2/IFN-γR2/BFP (FIG. 4, right panels; excited at 380 nm). Identical spectral signatures were observed in cells expressing IFN-γR1/EBFP and IFN-γR2/BFP (data not shown). Thus, the expected emission spectra are correct and all the constructs tested were functional.

Results

The carboxyl terminal end of the intracellular domain of the two chains of the human IFN-γ receptor complex, Hu-IFN-γR1 and Hu-IFN-γR2, were fused to BFP and GFP, respectively (FIG. 2). In addition, the carboxyl terminal end of the intracellular domain of the IL-10R2 chain was fused to GFP as a control. To ascertain that the receptor chains with the GFP and BFP (FIG. 3, right panel) were functional, these chains were transfected into Chinese hamster ovary q3 (CHO q3) cells to determine if they could function as well as the same chains without the fluorescent proteins (FIG. 3, left panel). The results show that MHC class I antigen induction in response to human IFN-γ was as effective as that of hamster IFN-γ demonstrating that the receptor chains with or without GFP and EBFP were functional. Cells were transfected with the IL-10R2/IFN-γR2/BFP and IFN-γR2/GFP separately to establish that the chains fused to BFP and GFP, respectively, provided the expected fluorescent signatures. The data showed that both IL-10R2/IFN-γR2/BFP and IFN-γR2/GFP provided the expected spectral signatures (FIG. 4). There was substantial background fluorescence when BFP was excited at 380 nm so that we used two-photon excitation (760 nm) to minimize this in the subsequent FRET assays.

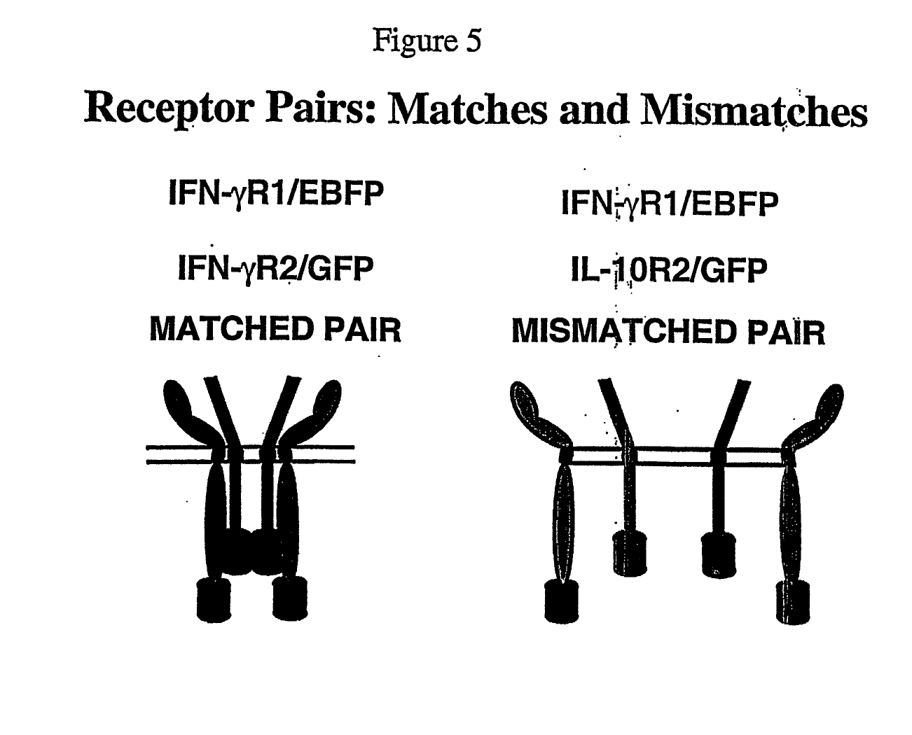

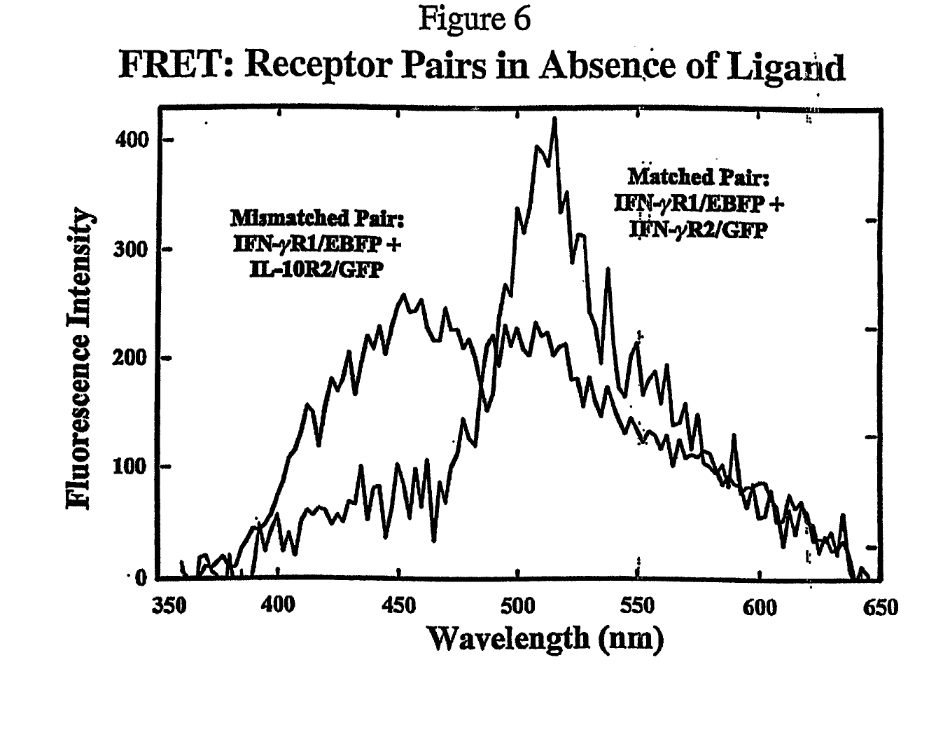

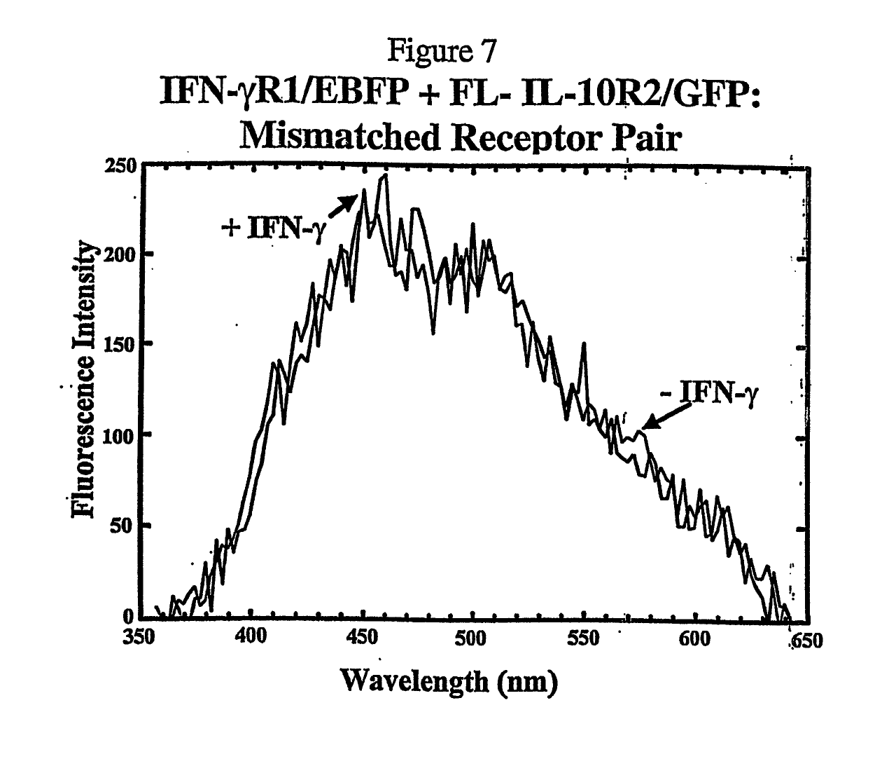

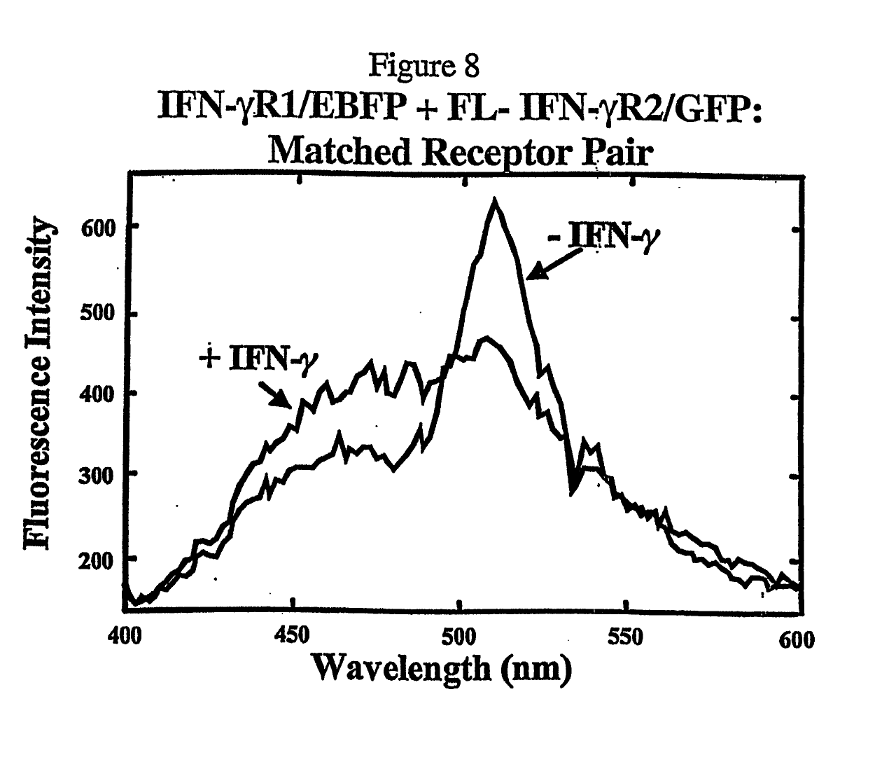

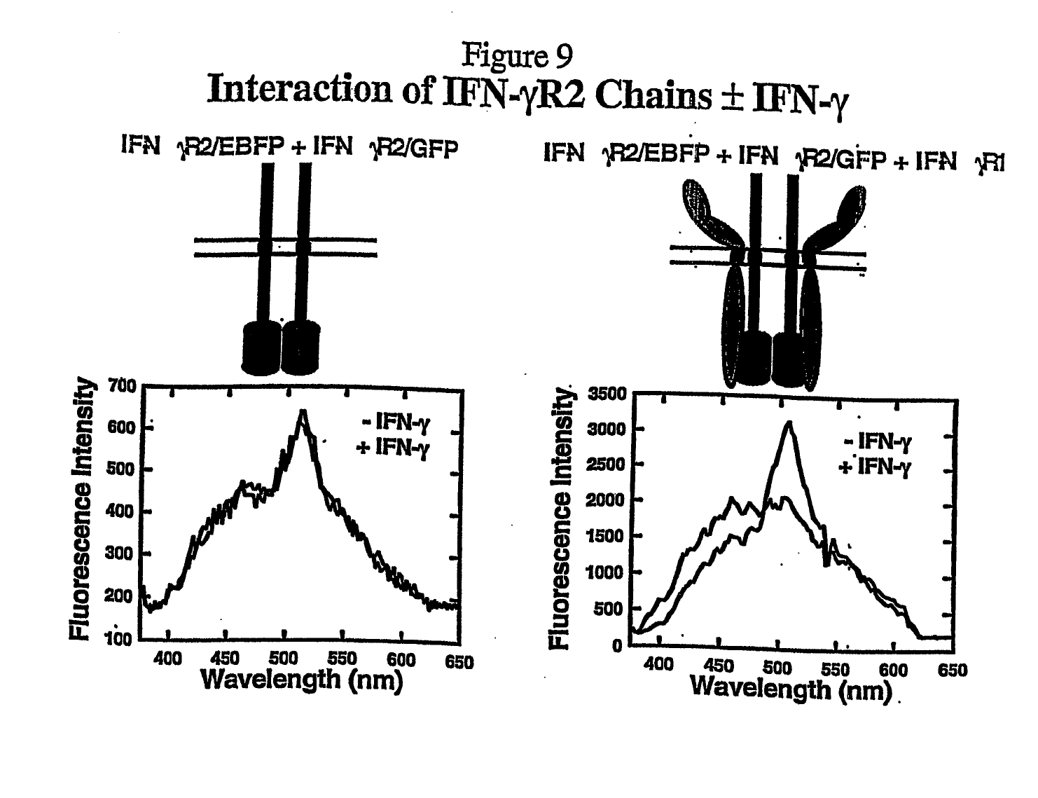

We next prepared transient transfectants in COS-1 cells expressing matched (Hu-IFN-γR1 and Hu-IFN-γR2) and mismatched (Hu-IFN-γR1 and Hu-IL-10R2) receptor pairs (FIG. 5). The matched pair, Hu-IFN-γR1/BFP and Hu-IFN-γR2/GFP, represents the two chains of the HU-IFN-γ receptor complex. The mismatched pair represents two chains from similar receptor complexes, Hu-IFN-γR1/BFP and Hu-IL-10R2/GFP, but are not known to interact. FRET was measured upon excitation at 760 nm in cells expressing each of these pairs of receptor chains in the absence of ligand (FIG. 6). The spectrum of cells expressing the mismatched pair Hu-IFN-γRI /BFP and Hu-IL-10R2/GFP shows the blue (EBFP) spectrum together with background fluorescence, demonstrating little or no interaction of these receptor chains. In contrast, the matched pair, Hu-IFN-γR1/EBFP and Hu-IFN-γR2/GFP, excited at 760 nm exhibits the fluorescence emission signature of the GFP demonstrating clear transfer between the BFP and GFP proteins. The distance between the intracellular regions of IFN-γR1/EBFP and IFN-γR2/GFP chains was calculated (89) to be 36 A whereas the distance between the Hu-IFN-γR1/EBFP and Hu-IL-10R2/GFP was not measurable. Thus, the IFN-γR1 and IFN-γR2 chains are preassociated prior to ligand binding. Addition of the ligand IFN-γ to cells expressing the mismatched pair IFN-γR1/EBFP and IL-10R2/GFP did not affect the spectrum (FIG. 7). The spectra were virtually identical in the presence or absence of IFN-γ. In contrast, the effect of IFN-γ on the FRET of the matched receptor pair expressed in cells showed that IFN-γ produced a change in the spectrum, causing a major reduction in the FRET compared to the FRET in the absence of IFN-γ (FIG. 8). The distance between the intracellular regions of Hu-IFN-γR1/EBFP and Hu-IFN-γR2/GFP chains in the absence and presence of ligand was calculated (89) to be 36 Δ and 63 Δ, respectively. Therefore, the intracellular domains of the IFN-γR1/BFP and IFN-γR2/GFP chains move apart on addition of ligand.

Because the IFN-γ receptor complex consists of four chains, we examined the effect of ligand on cells expressing only the IFN-γR2 chain (IFN-γR2/EBFP and IFN-γR2/GFP), but no IFN-γR1 chain, in the presence and absence of ligand (FIG. 9, left panels). In the absence of ligand, FRET was demonstrated between these two IFN-γR2 chains. There was no change in the spectrum in the presence of IFN-γ. This demonstrated that the IFN-γR2 chains are preassociated even in the absence of the IFN-γR1 chains. Furthermore, this observation that IFN-γ did not alter the spectrum and, therefore, the distance between the IFN-γR2 chains, is consistent with previous experiments that showed that IFN-γ did not produce any measurable signal transduction in cells expressing only the IFN-γR2 chain (90-95). When the spectra were measured in cells expressing both IFN-γR2/EBFP and IFN-γR2/GFP chains together with the IFN-γR1 chain without a fluorescent tag (FIG. 9, right panels), it was apparent that addition of the ligand IFN-γ changed the spectrum. In the absence of ligand, the spectrum was qualitatively similar in cells expressing IFN-γR2/EBFP and IFN-γR2/GFP whether the IFN-γR1 chain was present or not (cf., FIG. 9, left and right panels). However, in the presence of IFN-γ there was a large change in the spectrum of cells expressing IFN-γR2/EBFP, IFN-γR2/GFP and IFN-γR1 chains (FIG. 9, right panel). The results demonstrate that the IFN-γR2 chains are preassociated independent of the presence of the IFN-γR1 chain, but that the IFN-γR1 chain is required for the IFN-γR2 chains to move apart in response to IFN-γ. This result is consistent with previous reports that demonstrated that both chains are required for signal transduction (12, 90-97). However, it appears that in the presence of the IFN-γR1 chain, the IFN-γR2/EBFP and IFN-γR2/GFP chains are somewhat closer because there is a quantitative increase in FRET in the presence of the IFN-γR1 chain (cf., FIG. 9, left and right panels).

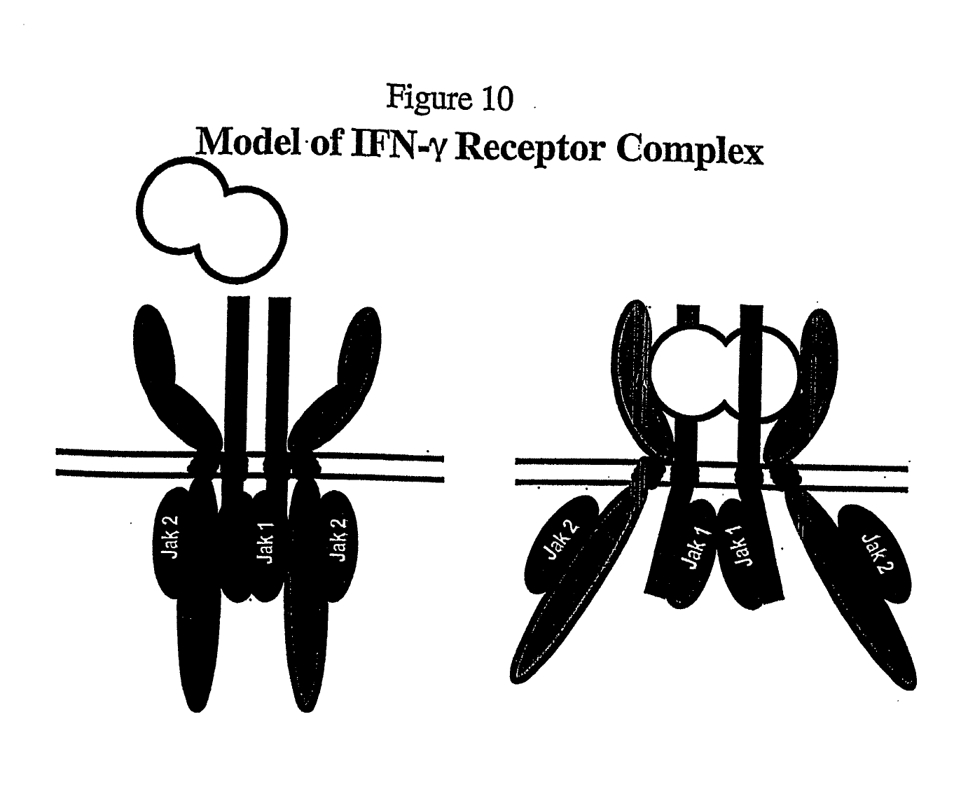

The model of the IFN-γ receptor complex consistent with the data is shown in FIG. 10. In the absence of ligand (FIG. 10, left panel), the intracellular domains of the receptor chains are close, preventing signal transduction as the intracellular components necessary for signaling are prevented from entering the receptor complex by the close proximity of the intracellular domains of these chains. Upon binding of the ligand IFN-γ, the intracellular domains of the receptor chains move apart, opening the area for multiple molecules required for signal transduction to enter the receptor complex (FIG. 9, right panel).

Discussion

The results demonstrate directly that the receptor chains of the IFN-γ receptor complex are preassociated on the cell membrane. Furthermore, the data prove that the receptor complex consists of four (or possibly more chains) because both the IFN-γR1 and IFN-γR2 pair (FIG. 6, FIG. 8) and the IFN-γR2 and IFN-γR2 pair (FIG. 9) are preassociated. It is also evident from the data that the IFN-γR2 and IFN-γR2 chains are preassociated in the absence of the IFN-γR1 chain, but that the presence of the IFN-γR1 brings the IFN-γR2 and IFN-γR2 chains closer together (FIG. 9). IFN-γ has no effect on the separation of IFN-γR2 chains in the absence of the IFN-γR I chain (FIG. 9, left panel) in accord with previous reports that IFN-γ does not bind to the IFN-γR2 chain and that IFN-γ exhibits no activity in the presence of the IFN-γR2 chain only (40, 42, 78, 87, 98-100). We suggest that this paradigm is likely to be applicable to other receptor chains and that receptor chains are preassociated in cells ready for activation by ligand. This conclusion leads to the deduction that cell surface receptor chains have specific sites, receptor association regions, RAR, that enable them to associate. Moreover, the increase in distance between the intracellular regions of the receptor chains opens the receptor complexes for the attachment of the signal transduction components that are excluded from the receptor complexes until ligand engagement. Thus, our direct measurements of the distances between the IFN-γ receptor chains before and after engagement of ligand lead to a new paradigm for receptor structure.

Because FRET is a real-time technique, changes in protein:protein interactions or protein positions in complexes in live cells can be determined over the course of time or modulation of the system being analyzed. Because relatively little is known about the physical association of specific receptor chains of multichain complexes and whether chains from one receptor type directly interact with another (12), the use of FRET to determine this should provide an answer these questions by applying the technique to cells. Furthermore, such studies can be extended to the downstream signal transduction events in response to ligand by determining the order, interactions and kinetics of these processes. This information will permit the development of pharmaceuticals that can interfere with IFN-γ signaling by interfering with IFN-γ binding to the complex, by blocking the activation of the ligand:receptor complex, or by altering downstream signal transduction events. The protein pairs used in the FRET assays would be adjusted accordingly to measure these specific events. In addition, this technology can be adapted to screen small molecule drugs by a quantitative, sensitive, and rapid real-time assay. A major goal will be use this technology to study the interaction of receptor chains and of many other proteins in cells under physiological conditions. Ultimately, this strategy could permit delineation of the entire proteome map of any cell, the interactome.

The ability to accomplish this efficiently will have major ramifications for analyzing protein-protein interactions, ligand-receptor interactions, signal transduction and many other cellular processes. Furthermore, questions relevant to protein interactions in cells that have been addressed indirectly should be able to be answered definitively with direct measurements and the time course of the events determined. The overall objective of our efforts is to provide an improved method to detect protein-protein interactions under physiological conditions in live cells by using FRET and to determine effects of ligands and other agents on the interactions by examining the IFN-γ receptor complex in detail by FRET. The results will have many useful applications in understanding receptor structure and provide a basis for high throughput screening of receptor ligands in single living cells.

### REFERENCE LIST

1. Fields, S., Song, O. (1989) A novel genetic system to detect protein-protein interactions. Nature 340, 245-246.

2. Aronheim, A., Zandi, E., Hennemann, H., Elledge, S. J., Karin, M. (1997) Isolation of an AP-1 repressor by a novel method for detecting protein-protein interactions. Mol. Cell Biol. 17, 3094-3102.

3. Bartel, P., Chien, C. T., Sternglanz, R., Fields, S. (1993) Elimination of false positives that arise in using the two-hybrid system. Biotechniques 14, 920-924.

4. Durfee, T., Becherer, K., Chen, P. -L., Yeh, S. -H., Yang, Y., Kilburn, A., Lee, W. -H., Elledge, S. J. (1993) The retinoblastoma protein associates with the protein phosphatase type I catalytic subunit. Genes and Devel. 7, 555-569.

5. Hardy, C. F., Sussel, L., Shore, D. (1992) A RAPI-interacting protein involved in transcriptional silencing and telomere length regulation. Genes Dev. 6, 801-814.

6. Luban, J., Bossolt, K. L., Franke, E. K., Kalpana, G. V., Goff, S. P. (1993) Human immunodeficiency virus type 1 Gag protein binds to cyclophilins A and B. Cell 73, 1067-1078.

7. Vojtek A. B., Hollenberg, S. M., Cooper, J. A. (1993) Mammalian Ras Interacts Directly with the Serine/Threonine Kinase Raf. Cell 74, 205-214.

8. Yang, X., Hubbard, E. J. -A., Carlson, M. (1992) A Protein Kinase Substrate Identified by the Two-Hybrid System. Science 257, 680-682.

9. Pollack, B. P., Kotenko, S. V., He, W., Izotova, L. S., Barnoski, B. L., Pestka, S. (1999) The human homologue of the yeast proteins Skb1 and Hsl7p interacts with Jak kinases and contains protein methyltransferase activity. J. Biol. Chem. 274, 31531-31542.

10. Sarkar, S., Pollack, B. P., Lin, K. T., Kotenko, S. V., Cook, J. R., Lewis, A., Pestka, S. (2001) hTid-1, a Human DnaJ Protein, Modulates the Interferon Signaling Pathway. J. Biol. Chem. 276, 49034-49042.

11. Langlois, R., Lee, C. C., Cantor, C. R., Vince, R., Pestka, S. (1976) The Distance Between Two Functionally Significant Regions of the 50S Escherichia coli Ribosome: The Erythromycin Binding Site and Proteins L7/L12. J. Mol. Biol. 106, 297-313.

12. Pestka, S., Kotenko, S. V., Muthukumaran, G., Izotova, L. S., Cook, J. R., Garotta, G. (1997) The interferon gamma (IFN-gamma) receptor: a paradigm for the multichain cytokine receptor. Cytokine Growth Factor. Rev. 8, 189-206.

13. Broudy, V. C., Lin, N. L., Buhring, H. J., Komatsu, N., Kavanagh, T. J. (1998) Analysis of c-kit receptor dimerization by fluorescence resonance energy transfer. Blood 91, 898-906.

14. Damjanovich, S., Bene, L., Matko, J., Alileche, A., Goldman, C. K., Sharrow, S., Waldmann, T. A. (1997) Preassembly of interleukin 2 (IL-2) receptor subunits on resting Kit 225 K6 T cells and their modulation by IL-2, IL-7, and IL-15: a fluorescence resonance energy transfer study. Proc. Natl. Acad. Sci. U.S.A 94, 13134-13139.

15. Guo, C., Dower, S. K., Holowka, D., Baird, B. (1995) Fluorescence resonance energy transfer reveals interleukin (IL)-1-dependent aggregation of IL-1 type I receptors that correlates with receptor activation. J. Biol. Chem. 270, 27562-27568.

16. Kenworthy, A. K., Edidin, M. (1998) Distribution of a glycosylphosphatidylinositol-anchored protein at the apical surface of MDCK cells examined at a resolution of <100 A using imaging fluorescence resonance energy transfer. J Cell Biol. 142, 69-84.

17. Heim, R., Prasher, D. C., Tsien, R. Y. (1994) Wavelength mutations and posttranslational autoxidation of green fluorescent protein. Proc. Natl. Acad. Sci. U.S.A 91, 12501-12504.

18. Prasher, D. C. (1995) Using GFP to see the light. Trends Genet. 11, 320-323.

19. Day, R. N. (1998) Visualization of Pit-I transcription factor interactions in the living cell nucleus by fluorescence resonance energy transfer microscopy. Mol. Endocrinol. 12, 1410-1419.

20. Gordon, G. W., Berry, G., Liang, X. H., Levine, B., Herman, B. (1998) Quantitative fluorescence resonance energy transfer measurements using fluorescence microscopy. Biophys. J 74, 2702-2713.

21. Janetopoulos, C., Jin, T., Devreotes, P. (2001) Receptor-mediated activation of heterotrimeric G-proteins in living cells. Science 291, 2408-2411.

22. Li, H. Y., Ng, E. K., Lee, S. M., Kotaka, M., Tsui, S. K., Lee, C. Y., Fung, K. P., Waye, M. M. (2001) Protein-protein interaction of FHL3-with FHL2 and visualization of their interaction by green fluorescent proteins (GFP) two-fusion fluorescence resonance energy transfer (FRET). J. Cell Biochem. 80, 293-303.

23. Llopis, J., Westin, S., Ricote, M., Wang, Z., Cho, C. Y., Kurokawa, R., Mullen, T. M., Rose, D. W., Rosenfeld, M. G., Tsien, R. Y., Glass, C. K., Wang, J. (2000) Ligand-dependent interactions of coactivators steroid receptor coactivator-1 and peroxisome proliferator-activated receptor binding protein with nuclear hormone receptors can be imaged in live cells and are required for transcription. Proc. Natl. Acad. Sci. U.S.A 97, 4363-4368.

24. Mahajan, N. P., Linder, K., Berry, G., Gordon, G. W., Heim, R., Herman, B. (1998) Bcl-2 and Bax interactions in mitochondria probed with green fluorescent protein and fluorescence resonance energy transfer. Nat. Biotechnol. 16, 547-552.

25. Margolin, W. (2000) Green fluorescent protein as a reporter for macromolecular localization in bacterial cells. Methods 20, 62-72.

26. Mizuno, H., Sawano, A., Eli, P., Hama, H., Miyawaki, A. (2001) Red fluorescent protein from Discosoma as a fusion tag and a partner for fluorescence resonance energy transfer. Biochemistry 40, 2502-2510.

27. Overton, M. C., Blumer, K. J. (2000) G-protein-coupled receptors function as oligomers in vivo. Curr. Biol. 10, 341-344.

28. Pollok, B. A., Heim, R. (1999) Using GFP in FRET-based applications. Trends Cell Biol. 9, 57-60.

29. Prufer, K., Racz, A., Lin, G. C., Barsony, J. (2000) Dimerization with retinoid λ receptors promotes nuclear localization and subnuclear targeting of vitamin D receptors. J. Biol. Chem. 275, 41114-41123.

30. Schmid, J. A., Birbach, A., Hofer-Warbinek, R., Pengg, M., Burner, U., Furtmuller, P. G., Binder, B. R., de Martin, R. (2000) Dynamics of NF kappa B and Ikappa Balpha studied with green fluorescent protein (GFP) fusion proteins. Investigation of GFP-p65 binding to DNa by fluorescence resonance energy transfer. J. Biol. Chem. 275, 17035-17042.

31. Sorkin, A., McClure, M., Huang, F., Carter, R. (2000) Interaction of EGF receptor and grb2 in living cells visualized by fluorescence resonance energy transfer (FRET) microscopy. Curr. Biol. 10, 1395-1398.

32. Vanderklish, P. W., Krushel, L. A., Holst, B. H., Gally, J. A., Crossin, K. L., Edelman, G. M. (2000) Marking synaptic activity in dendritic spines with a calpain substrate exhibiting fluorescence resonance energy transfer. Proc. Natl. Acad. Sci. U.S.A 97, 2253-2258.

33. Xia, Z., Zhou, Q., Lin, J., Liu, Y. (2001) Stable SNARE complex prior to evoked synaptic vesicle fusion revealed by fluorescence resonance energy transfer. J. Biol. Chem. 276, 1766-1771.—

34. Harpur, A. G., Wouters, F. S., Bastiaens, P. I (2001) Imaging FRET between spectrally similar GFP molecules in single cells. Nat. Biotechnol. 19, 167-169.

35. Mochizuki, N., Yamashita, S., Kurokawa, K., Ohba, Y., Nagai, T., Miyawaki, A., Matsuda, M. (2001) Spatio-temporal images of growth-factor-induced activation of Ras and Rap1. Nature 411, 1065-1068.

36. Langer, J. A., Pestka, S. (1988) Interferon receptors. Immunol. Today 9, 393-400.

37. Pestka, S., Langer, J. A., Zoon, K. C., Samuel, C. E. (1987) Interferons and their actions. Annu. Rev. Biochem. 56, 727-777.

38. Pestka, S. (1997) The Interferon Receptors. Seminars in Oncology 24, Suppl 9, S9-18-S9-40.

39. Mariano, T. M., Kozak, C. A., Langer, J. A., Pestka, S. (1987) The mouse immune interferon receptor gene is located on chromosome 10. J. Biol. Chem. 262, 5812-5814.

40. Rashidbaigi, A., Langer, J. A., Jung, V., Jones, C., Morse, H. G., Tischfield, J. A., Trill, J. J., Kung, H. -F., Pestka, S., Kung, H. F. (1986) The gene for the human immune interferon receptor is located on chromosome 6. Proc.Natl.Acad.Sci. U.S.A. 83, 384-388.

41. Hibino, Y., Mariano, T. M., Kumar, C. S., Kozak, C. A., Pestka, S. (1991) Expression and Reconstitution of a Biologically Active Mouse Interferon Gamma Receptor in Hamster Cells: Chromosomal Location of an Accessory Factor. J. Biol. Chem. 266, 6948-6951.

42. Jung, V., Rashidbaigi, A., Jones, C., Tischfield, J. A., Shows, T. B., Pestka, S. (1987) Human Chromosomes 6 and 21 are Required for Sensitivity to Human Interferon Gamma. Proc. Natl. Acad. Sci. U.S.A. 84, 4151-4155.

43. Jung, V., Jones, C., Rashidbaigi, A., Geyer, D. D., Morse, H. G., Wright, R. B., Pestka, S. (1988) Chromosome Mapping of Biological Pathways by Fluorescence-activated Cell Sorting and Cell Fusion: The Human Interferon Gamma Receptor as a Model System. Somat. Cell Mol. Genet. 14, 583-592.

44. Youssoufian, H., Longmore, G., Neumann, D., Yoshimura, A., Lodish, H. F. (1993) Structure, function, and activation of the erythropoietin receptor. Blood 81, 2223-2236.

45. Lemmon, M. A., Bu, Z., Ladbury, J. E., Zhou, M., Pinchasi, D., Lax, I., Engelman, D. M., Schlessinger, J. (1997) Two EGF molecules contribute additively to stabilization of the EGFR dimer. EMBO J. 16, 281-294.

46. Watowich, S. S., Yoshimura, A., Longmore, G. D., Hilton, D. J., Yoshimura, Y., Lodish, H. F. (1992) Homodimerization and constitutive activation of the erythropoietin receptor. Proc. Natl. Acad. Sci. U.S.A 89, 2140-2144.

47. Watowich, S. S., Hilton, D. J., Lodish, H. F. (1994) Activation and inhibition of erythropoietin receptor function: role of receptor dimerization. Mol. Cell Biol. 14, 3535-3549.

48. Yarden, Y., Schlessinger, J. (1987) Self-phosphorylation of epidermal growth factor receptor: evidence for a model of intermolecular allosteric activation. Biochemistry 26, 1434-1442.

49. Yarden, Y., Schlessinger, J. (1987) Epidermal growth factor induces rapid, reversible aggregation of the purified epidermal growth factor receptor. Biochemistry 26, 1443-1451.

50. Zhou, M., Felder, S., Rubinstein, M., Hurwitz, D. R., Ullrich, A., Lax, I., Schlessinger, J. (1993) Real-time measurements of kinetics of EGF binding to soluble EGF receptor monomers and dimers support the dimerization model for receptor activation. Biochemistry 32, 8193-8198.

51. Baer, K., A I Hasani, H., Parvaresch, S., Corona, T., Rufer, A., Nolle, V., Bergschneider, E., Klein, H. W. (2001) Dimerization-induced activation of soluble insulin/IGF-1 receptor kinases: an alternative mechanism of activation. Biochemistry 40, 14268-14278.

52. Bishayee, S., Majumdar, S., Khire, J., Das, M. (1989) Ligand-induced dimerization of the platelet-derived growth factor receptor. Monomer-dimer interconversion occurs independent of receptor phosphorylation. J. Biol. Chem. 264, 11699-11705.

53. Bouvier, M. (2001) Oligomerization of G-protein-coupled transmitter receptors. Nat. Rev. Neurosci. 2, 274-286.

54. Brennan, P. J., Kumagai, T., Berezov, A., Murali, R., Greene, M. I., Kumogai, T. (2000) HER2/neu: mechanisms of dimerization/oligomerization. Oncogene 19, 6093-6101.

55. Chan, F. K., Chun, H. J., Zheng, L., Siegel, R. M., Bui, K. L., Lenardo, M. J. (2000) A domain in TNF receptors that mediates ligand-independent receptor assembly and signaling. Science 288, 2351-2354.

56. Chow, D., Ho, J., Nguyen Pham, T. L., Rose-John, S., Garcia, K. C. (2001) In vitro reconstitution of recognition and activation complexes between interleukin-6 and gp130. Biochemistry 40, 7593-7603.

57. Dcvi, L. A. (2001) Heterodimerization of G-protein-coupled receptors: pharmacology, signaling and trafficking. Trends Pharmacol. Sci. 22, 532-537.

58. Gadella, T. W., Jr., Jovin, T. M. (1995) Oligomerization of epidermal growth factor receptors on A431 cells studied by time-resolved fluorescence imaging microscopy. A stereochemical model for tyrosine kinase receptor activation. J. Cell Biol. 129, 1543-1558.

59. Gamett, D. C., Pearson, G., Cerione, R. A., Friedberg, 1. (1997) Secondary dimerization between members of the epidermal growth factor receptor family. J. Biol. Chem. 272, 12052-12056.

60. Guimond, A., Sulea, T., Zwaagstra, J. C., Ekiel, I., O'Connor-McCourt, M. D. (2002) Identification of a functional site on the type I TGF-beta receptor by mutational analysis of its ectodomain. FEBS Lett. 513, 147-152.

61. Jiang, G., Hunter, T. (1999) Receptor signaling: when dimerization is not enough. Curr. Biol. 9, R568-R571.

62. Johannessen, L. E., Haugen, K. E., ostvold, A. C., Stang, E., Madshus, I. H. (2001) Heterodimerization of the epidermal-growth-factor (EGF) receptor and ErbB2 and the affinity of EGF binding are regulated by different mechanisms. Biochem. J. 356, 87-96.

63. Kan, M., Wu, X., Wang, F., McKeehan, W. L. (1999) Specificity for fibroblast growth factors determined by heparan sulfate in a binary complex with the receptor kinase. J. Biol. Chem. 274, 15947-15952.

64. Maher, D. W., Strawn, L. M., Donoghue, D. J. (1993) Alanine mutagenesis of conserved residues in the platelet-derived growth factor family: identification of residues necessary for dimerization and transformation. Oncogene 8, 533-541.

65. Martin-Fernandez, M., Clarke, D. T., Tobin, M. J., Jones, S. V., Jones, G. R. (2002) Preformed Oligomeric Epidermal Growth Factor Receptors Undergo an Ectodomain Structure Change during Signaling. Biophys. J. 82, 2415-2427.

66. McInnes, C., Sykes, B. D. (1997) Growth factor receptors: structure, mechanism, and drug discovery. Biopolymers 43, 339-366.

67. Moriki, T., Maruyama, H., Maruyama, I. N. (2001) Activation of preformed EGF receptor dimers by ligand-induced rotation of the transmembrane domain. J. Mol. Biol. 311, 1011-1026.

68. Morrow, M. R., Grant, C. W. (2000) The EGF receptor transmembrane domain: peptide-peptide interactions in fluid bilayer membranes. Biophys. J 79, 2024-2032.

69. Sako, Y., Minoghchi, S., Yanagida, T. (2000) Single-molecule imaging of EGFR signalling on the surface of living cells. Nat. Cell Biol. 2, 168-172.

70. Siegel, R. M., Frederiksen, J. K., Zacharias, D. A., Chan, F. K., Johnson, M., Lynch, D., Tsien, R. Y., Lenardo, M. J. (2000) Fas preassociation required for apoptosis signaling and dominant inhibition by pathogenic mutations. Science 288, 2354-2357.

71. van den, A. F. (2001) Structural insights into the ligand binding domains of membrane bound guanylyl cyclases and natriuretic peptide receptors. J. Mol. Biol. 311, 923-937.

72. Wang, F., Kan, M., McKeehan, K., Jang, J. H., Feng, S., McKeehan, W. L. (1997) A homeo-interaction sequence in the ectodomain of the fibroblast growth factor receptor. J. Biol. Chem. 272, 23887-23895.

73. Watowich, S. S. (1999) Activation of erythropoietin signaling by receptor dimerization. Int. J. Biochem. Cell Biol. 31, 1075-1088.

74. Wiesmann, C., Muller, Y. A., de Vos, A. M. (2000) Ligand-binding sites in Ig-like domains of receptor tyrosine kinases. J. Mol. Med. 78, 247-260.

75. Wilson, I. A., Jolliffe, L. K. (1999) The structure, organization, activation and plasticity of the erythropoietin receptor. Curr. Opin. Struct. Biol. 9, 696-704.

76. Wiseman, P. W., Petersen, N, O. (1999) Image correlation spectroscopy. II. Optimization for ultrasensitive detection of preexisting platelet-derived growth factor-beta receptor oligomers on intact cells. Biophys. J 76, 963-977.

77. Aguet, M., Dembic, Z., Merlin, G. (1988) Molecular Cloning and Expression of the Human Interferon-gamma Receptor. Cell 55, 273-280.

78. Soh, J., Donnelly, R. J., Kotenko, S., Mariano, T. M., Cook, J. R., Wang, N., Emanuel, S. L., Schwartz, B., Miki, T., Pestka, S. (1994) Identification and Sequence of an Accessory Factor Required for Activation of the Human Interferon Gamma Receptor. Cell 76, 793-802.

79. Darnell, J. E., Jr., Kerr, 1. M., Stark, G. R. (1994) Jak-STAT Pathways and Transcriptional Activation in Response to IFNs and Other Extracellular Signaling Proteins. Science 264, 1415-1421.

80. Bach, E. A., Tanner, J. W., Marsters, S., Ashkenazi, A., Aguet, M., Shaw, A. S., Schreiber, R. D. (1996) Ligand-induced assembly and activation of the gamma interferon receptor in intact cells. Mol. Cell. Biol. 16, 3214-3221.

81. Ishiharu, C., Ochiai, K., Kagami, M., Matsuyama, G., Koya, N., Tomioka, H. (1998) Interferon-gamma receptor beta-chain expression and formation of alpha- and beta-chain complexes after receptor conjugation on human peripheral eosinophils. Int. Arch. Allergy Immunol. 117 Suppl 1, 72-76.

82. Kotenko, S. V., Izotova, L. S., Pollack, B. P., Mariano, T. M., Donnelly, R. J., Muthukumaran, G., Cook, J. R., Garotta, G., Silvennoinen, O., IhIe, J. N., Pestka, S. (1995) Interaction Between the Components of the Interferon Gamma Receptor Complex. J. Biol. Chem. 270, 20915-20921.

83. Kotenko, S. V., Izotova, L. S., Pollack, B. P., Muthukumaran, G., Paukku, K., Silvennoinen, O., Ihle, J. N., Pestka, S. (1996) Other Kinases can Substitute for Jak2 in Signal Transduction by IFN-Gamma. J. Biol. Chem. 271, 17174-17182.

84. Kotenko, S. V., Krause, C. D., Izotova, L. S., Pollack, B. P., Wu, W., Pestka, S. (1997) Identification and functional characterization of a second chain of the interleukin-10 receptor complex. EMBO J. 16, 5894-5903.

85. Heim, R., Cubitt, A. B., Tsien, R. Y. (1995) Improved green fluorescence. Nature 373, 663-664.

86. Heim, R., Tsien, R. Y. (1996) Engineering green fluorescent protein for improved brightness, longer wavelengths and fluorescence resonance energy transfer. Curr. Biol. 6, 178-182.

87. Jung, V., Jones, C., Kumar, C. S., Stefanos, S., O'Connell, S., Pestka, S. (1990) Expression and Reconstitution of a Biologically Active Human Interferon Gamma Receptor in Hamster Cells. J. Biol. Chem. 265, 1827-1830.

88. Ormo, M., Cubitt, A. B., Kallio, K., Gross, L. A., Tsien, R. Y., Remington, S. J. (1996) Crystal structure of the Aequorea victoria green fluorescent protein. Science 273, 1392-1395.

89. Langlois, R., Lee, C. C., Cantor, C. R., Vince, R., Pestka, S. (1976) The Distance Between Two Functionally Significant Regions of the 50S Escherichia coli Ribosome: The Erythromycin Binding Site and Proteins L7/L12. J. Mol. Biol. 106, 297-313.

90. Jung, V., Rashidbaigi, A., Jones, C., Tischfield, J. A., Shows, T. B., Pestka, S. (1987) Human Chromosomes 6 and 21 are Required for Sensitivity to Human Interferon Gamma. Proc. Natl. Acad. Sci. U.S.A. 84, 4151-4155.

91. Jung, V., Jones, C., Rashidbaigi, A., Geyer, D. D., Morse, H. G., Wright, R. B., Pestka, S. (1988) Chromosome Mapping of Biological Pathways by Fluorescence-activated Cell Sorting and Cell Fusion: The Human Interferon Gamma Receptor as a Model System. Somat. Cell Mol. Genet. 14, 583-592.

92. Jung, V., Jones, C., Kumar, C. S., Stefanos, S., O'Connell, S., Pestka, S. (1990) Expression and Reconstitution of a Biologically Active Human Interferon Gamma Receptor in Hamster Cells. J. Biol. Chem. 265, 1827-1830.

93. Soh, J., Donnelly, R. J., Kotenko, S., Mariano, T. M., Cook, J. R., Wang, N., Emanuel, S. L., Schwartz, B., Miki, T., Pestka, S. (1994) Identification and Sequence of an Accessory Factor Required for Activation of the Human Interferon Gamma Receptor. Cell 76, 793-802.

94. Lembo, D., Ricciardi, P., Alber, G., Ozmen, L., Landolfo, S., Bluthmann, H., Dembic, Z., Kotenko, S. V., Cook, J. R., Pestka, S., Garotta, G. (1996) Mouse Macrophages Carrying Both Subunits of the Human Interferon-gamma (IFN-gamma) Receptor Respond to Human IFN-gamma but Do Not Acquire Full Protection Against Viral Cytopathic Effect. J. Biol. Chem. 271, 32659-32666.

95. Hemmi, S., Bohni, R., Stark, G., Di Marco, F., Aguet, M. (1994) A Novel Member of the Interferon Receptor Family Complements Functionality of the Murine Interferon-Gamma Receptor in Human Cells. Cell 76, 803-810.

96. Rashidbaigi, A., Langer, J. A., Jung, V., Jones, C., Morse, H. G., Tischfield, J. A., Trill, J. J., Kung, H. -F., Pestka, S., Kung, H. F. (1986) The gene for the human immune interferon receptor is located on chromosome 6. Proc. Natl. Acad. Sci. U.S.A. 83, 384-388.

97. Pestka, S. (2000) The human interferon alpha species and receptors. Biopolymers 55, 254-287.

98. Cook, J. R., Emanuel, S. L., Donnelly, R. J., Soh, J., Mariano, T. M., Schwartz, B., Rhee, S., Pestka, S. (1994) Sublocalization of the Human Interferon-gamma Receptor Accessory Factor Gene and Characterization of Accessory Factor Activity by Yeast Artificial Chromosomal Fragmentation. J. Biol. Chem. 269, 7013-7018.

99. Hemmi, S., Bohni, R., Stark, G., Di Marco, F., Aguet, M. (1994) A novel member of the interferon receptor family complements functionality of the murine interferon gamma receptor in human cells. Cell 76, 803-810.

100. Soh, J., Donnelly, R. J., Mariano, T. M., Cook, J. R., Schwartz, B., Pestka, S. (1993) Identification of a Yeast Artificial Chromosome Clone Encoding an Accessory Factor for the Human Interferon Gamma Receptor: Evidence for Multiple Accessory Factors. Proc. Natl. Acad. Sci. U.S.A. 90, 8737-8741.

101. Barbosa, J. A., Kamarck, M. E., Biro, P. A., Weissman, S. M., Ruddle, F. H. (1982) Identification of human genomic clones coding the major histocompatibility antigens HLA-a2 and HLA-B7 by DNA-mediated gene transfer. Proc. Natl. Acad. Sci. U.S.A 79, 6327-6331.

All of the above-cited references and publications are hereby incorporated by reference.

Equivalents

Those skilled in the art will recognize, or be able to ascertain using no more than routine experimentation, numerous equivalents to the specific polypeptides, nucleic acids, methods, assays and reagents described herein. Such equivalents are considered to be within the scope of this invention and are covered by the following claims.

1. A method to detect quaternary structural change in a multi-component membrane complex upon binding of an analyte, comprising:
(a) providing a multi-component membrane complex which binds an analyte, comprising:
(i) a first fusion protein comprising a first polypeptide and a donor fluorescent protein moiety, wherein the donor fluorescent protein moiety, when excited at a first excitation wavelength, fluoresces at a first emission wavelength;
(ii) a second fusion protein comprising a second polypeptide and an acceptor fluorescent protein moiety, wherein the acceptor fluorescent protein moiety, when excited by said first emission wavelength, fluoresces at a second emission wavelength;
wherein in the absence of said analyte, said donor and acceptor fluorescent protein moieties are sufficiently close for FRET (fluorescent resonant energy transfer) to occur, and excitation with light at said first excitation wavelength produces emission at said second emission wavelength;
(b) exciting said donor fluorescent protein moiety with said first excitation wavelength;
(c) contacting said multi-component membrane complex with said analyte;
(d) detecting emission of at least one of said first or second emission wavelength;
wherein a shift from emission at said second emission wavelength to said first emission wavelength indicates that a quaternary structural change occurs in said multi-component complex upon binding of said analyte.

2. The method of claim 1, wherein the multi-component membrane complex is a receptor.

3. The method of claim 2, wherein the receptor is a cytokine receptor.

4. The method of claim 3, wherein the cytokine receptor is IFN-gamma receptor.

5. The method of claim 2, wherein the multi-component membrane complex is: a growth factor receptor, a GPCR (G-Protein Coupled Receptor), an MIRR (Multisubunit Immune Recognition Receptor) receptor, or an orphan receptor.

6. The method of claim 1, wherein at least one of said first and second fusion proteins is a transmembrane protein.

7. The method of claim 1, wherein said donor and acceptor fluorescent protein moieties are Aequorea-related fluorescent protein moieties.

8. The method of claim 7, wherein said Aequorea-related protein moieties are selected from the proteins listed in Table I.

9. The method of claim 7, wherein said donor and acceptor fluorescent protein moieties are BFP and GFP, respectively.

10. The method of claim 9, wherein said donor fluorescent protein moiety is excited at a first excitation wavelength by two-photo excitation.

11. The method of claim 1, wherein the multi-component membrane complex is provided in a single live and intact cell.

12. The method of claim 11, wherein the multi-component membrane complex is provided as a plasma membrane complex in a single live and intact cell.

13. The method of claim 12, wherein said donor and acceptor fluorescent protein moieties are both intracellular or are both extracellular.

14. The method of claim 1, wherein the multi-component membrane complex is a multimeric complex containing more than one member for at least one type of subunits.

15. The method of claim 14, wherein said multi-component membrane complex contains more than one said first polypeptide, wherein one or more said first polypeptide is fused to said donor fluorescent protein moiety.

16. The method of claim 14, wherein said multi-component membrane complex contains more than one said second polypeptide, wherein one or more said second polypeptide is fused to said acceptor fluorescent protein moiety.

17. The method of claim 1, wherein said first and second polypeptides are the same.

18. The method of claim 1, wherein said first and second polypeptides are different.

19. The method of claim 1, wherein the first excitation wavelength is generated by a laser.

20. The method of claim 19, wherein the laser is generated from a confocal microscope.

21. The method of claim 1, wherein the first and second fusion proteins are provided by introducing into a cell nucleic acids that encode said first and second fusion proteins.

22. A method of identifying a ligand for a multi-component membrane complex, comprising:
(a) providing a multi-component membrane complex, comprising:
(i) a first fusion protein comprising a first polypeptide and a donor fluorescent protein moiety, wherein the donor fluorescent protein moiety, when excited at a first excitation wavelength, fluoresces at a first emission wavelength;
(ii) a second fusion protein comprising a second polypeptide and an acceptor fluorescent protein moiety, wherein the acceptor fluorescent protein moiety, when excited by said first emission wavelength, fluoresces at a second emission wavelength;
wherein in the absence of said analyte, said donor and acceptor fluorescent protein moieties are sufficiently close for FRET (fluorescent resonant energy transfer) to occur, and excitation with light at said first excitation wavelength produces emission at said second emission wavelength;
(b) exciting said donor fluorescent protein moiety with said first excitation wavelength;
(c) contacting said multi-component membrane complex with a test compound;
(d) detecting emission of at least one of said first or second emission wavelength;
(e) identifying a test compound as the ligand of said multi-component membrane complex if a shift from emission at said second emission wavelength to said first emission wavelength occurs.

23. The method of claim 22, wherein the multi-component membrane complex is provided in a single live and intact cell.

24. The method of claim 23, wherein the multi-component membrane complex is provided as a plasma membrane complex in a single live and intact cell.

25. The method of claim 24, wherein said donor and acceptor fluorescent protein moieties are both intracellular or are both extracellular.

26. The method of claim 24, further comprising carrying out steps (a)-(e) in a microtiter plate comprising 96 wells or 384 wells.

27. The method of claim 24, wherein step (e) is effected by a FACS (Fluorescent Activated Cell Sorter) machine.

28. The method of claim 22, wherein the multi-component membrane complex is a receptor.

29. The method of claim 28, wherein the receptor is a cytokine receptor.

30. The method of claim 29, wherein the cytokine receptor is IFN-gamma receptor.

31. The method of claim 28, wherein the receptor is: a growth factor receptor, a GPCR (G-Protein Coupled Receptor), an MIRR (Multisubunit Immune Recognition Receptor) receptor, or an orphan receptor.

32. The method of claim 22, wherein at least one of said first and second fusion proteins is a transmembrane protein.

33. The method of claim 22, wherein said donor and acceptor fluorescent protein moieties are Aequorea-related fluorescent protein moieties.

34. The method of claim 33, wherein said Aequorea-related protein moieties are selected from the proteins listed in Table I.

35. The method of claim 33, wherein said donor and acceptor fluorescent protein moieties are BFP and GFP, respectively.

36. The method of claim 35, wherein said donor fluorescent protein moiety is excited at a first excitation wavelength by two-photo excitation.

37. The method of claim 22, wherein the multi-component membrane complex is a multimeric complex containing more than one member for at least one type of subunits.

38. The method of claim 37, wherein said multi-component membrane complex contains more than one said first polypeptide, wherein one or more said first polypeptide is fused to said donor fluorescent protein moiety.

39. The method of claim 37, wherein said multi-component membrane complex contains more than one said second polypeptide, wherein one or more said second polypeptide is fused to said acceptor fluorescent protein moiety.

40. The method of claim 22, wherein said first and second polypeptides are the same.

41. The method of claim 22, wherein said first and second polypeptides are different.

42. The method of claim 22, wherein the first excitation wavelength is generated by a laser.

43. The method of claim 42, wherein the laser is generated from a confocal microscope.

44. The method of claim 22, wherein the first and second fusion proteins are provided by introducing into a cell nucleic acids that encode said first and second fusion proteins.

45. A method of conducting a pharmaceutical business, comprising:
(a) identifying, using the method of claim 22, a ligand for a multi-component membrane complex;
(b) conducting therapeutic profiling of said ligand identified in step (a), or further analogs thereof, for efficacy and toxicity in animals; and
(c) formulating a pharmaceutical preparation including one or more ligands identified in step (b) as having an acceptable therapeutic profile.

46. The method of claim 45, further comprising a step of establishing a distribution system for distributing the pharmaceutical preparation for sale.

47. The method of claim 45, further comprising a step of establishing a sales group for marketing the pharmaceutical preparation.

48. A method of conducting a target discovery business comprising:
(a) identifying, using the method of claim 22, a ligand for a multi-component membrane complex;
(b) (optionally) conducting therapeutic profiling of ligands identified in step (a) for efficacy and toxicity in animals; and
(c) licensing, to a third party, the rights for further drug development and/or sales for agents identified in step (a), or analogs thereof.

49. A ligand for a multi-component membrane complex identified by the method of claim 22.
